(19) United States
(12) Patent Application Publication
(10) Pub. No.: US 2022/0324813 A1
(43) Pub. Date: Oct. 13, 2022

(54) COMPOSITIONS AND METHODS FOR THE TREATMENT OF ANAL AND RECTAL DISORDERS

(71) Applicant: Cellix Bio Private Limited, Hyderabad (IN)

(72) Inventor: Mahesh Kandula, East Godavari District, Andhra Pradesh (IN)

(73) Assignee: Cellix Bio Private Limited, Hyderabad (IN)

(21) Appl. No.: 17/631,763

(22) PCT Filed: Jul. 16, 2020
(86) PCT No.: PCT/IB2020/056687

§ 371 Date: Jan. 31, 2022

(30) Foreign Application Priority Data
Jul. 30, 2019 (IN) 201941030819

(51) Int. Cl. C07D 233/08; C07D 409/12

(52) U.S. Cl. CPC C07D 233/08; C07D 409/12

(57) ABSTRACT

The present invention relates to compounds or its pharmaceutical acceptable polymorphs, solvates, enantiomers, stereoisomers, prodrugs, hydrates and or derivatives thereof. The Pharmaceutical composition comprises an effective amount of compounds of formula I, formula II, formula III, formula IV, Formula V, formula VI or formula VII and the methods of using the composition for treating disorder affecting the anus and rectum. The composition can be formulated for oral administration, rectal administration, topical administration, transmucosal, transdermal administration, spray, injection or other known formulation in the art.

### PRIORITY

The present application claims the benefit of Indian Provisional Application No. IN 201941030819 filed on 30 Jul. 2019, the contents of which are incorporated in its entirety herein by reference.

### FIELD OF THE INVENTION

This disclosure generally relates to compounds and compositions for the treatment of condition affecting anal and rectal region and its associated complications thereof. More particularly, this invention relates to treating subjects with a pharmaceutically acceptable dose of compounds, salts, crystals, solvates, enantiomer, stereoisomer, esters, hydrates, derivatives or mixtures thereof.

### BACKGROUND OF THE INVENTION

Anal and rectal disorders comprise of wide range of disorders mainly affecting the anal canal and rectum. Anal and rectal disorder includes hemorrhoids, abscesses, fistula, fissures, anal itching, proctalgia fugax, fecal incontinence (FI), and anorectal pain.

Fecal incontinence or Bowel incontinence, the recurrent uncontrolled passage of fecal material, affects many men and women and often is undertreated and underdiagnosed due to social stigmatization. Relatives unable to care for an older patient at home may find fecal incontinence a deciding factor for admitting the patient to a nursing home. Fecal incontinence is estimated to affect as many as 45% of residents in long-term care facilities.

Fecal incontinence results from injuries or diseases of the spinal cord, congenital abnormalities, accidental injuries to the rectum and anus, diabetes, severe dementia, fecal impaction, extensive inflammatory processes, tumors, and injuries to the anal sphincters.

Fecal incontinence can be divided into three subtypes with overlapping characteristics:

(i) Passive incontinence: defined by the involuntary loss of stool without patient awareness. This type of incontinence can be caused by weakness of the internal or external anal sphincter, neuropathy, loss of perception, or impairment of reflexes. Passive fecal incontinence is believed to be the result of sphincter injury secondary to vaginal delivery or due to forceps-assisted births.
(ii) Urge incontinence: the inability to retain fecal matter despite active attempts. This type typically is seen with rectal hypersensitivity or impaired rectal compliance. Damage to the external anal sphincter may manifest as urge incontinence, resulting in a diminished capacity and causing an increase in frequency.
(iii) Fecal seepage, or stool: that leaks after a normal bowel movement without patient awareness. This type is more common in men possibly due to paradoxical high sphincter resting pressures. Anal sphincter tone and function are typically normal, but diagnostic testing may show incomplete evacuation of stool and some impairment of rectal sensation.

Because fecal incontinence is complex, a detailed history and tailored diagnostic testing are needed to formulate a treatment plan for each patient. Risk factors for fecal incontinence include female sex, pregnancy, labor trauma, parity, perianal surgery, neurologic causes, obesity, cigarette smoking, diabetes, and COPD. Diarrhea is a common modifiable risk factor for fecal incontinence with an odds ratio of 53 (95% CI, 6.1-471), which contributes to urgency and increased frequency.

Managing acute pathology of often relies on the addressing underlying pathology and symptoms of the disease. There is currently a need in the art for new compositions to treat the anal and rectal conditions more special all types of fecal incontinence and its associated complications progression.

### SUMMARY OF THE INVENTION

The present invention provides compounds, compositions containing compounds of formula I, formula II, formula III, formula IV, formula V, formula VI, or formula VII and methods for using the same to treat, prevent and/or ameliorate the effects of the conditions related to anal and rectal disorders and associate complication and manifestation thereof.

Throughout the present disclosure,

RH independently represents:
NULL, Cl—, Na+, pyridoxine, R-lipoic acid, methyl cellulose, glutamic acid, aspartic acid, valproic acid, prostaglandin F2a, dopamine, morphine, loperamide, b-blockers, sodium valproate, codeine phosphate, N-acetyl cysteine, cysteine, 5-Hydroxytryptamine, Zn+2, Mg+2, Mn+2, I—, Ag+2, Na, K, mesalamine, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, galactaric acid, gentisic acid, glucoheptonic acid, gluconic acid, glucuronic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, n-acetyl cysteine (nac), furoate, methyl furoate, ethyl furoate, aminocaproic acid, caprilic acid, alpha lipoic acid, myristic acid, myristoleic acid, palmitoleic acid, elaidic acid, linoleic acid, linolenic acid, linolelaidic acid, arachidonic acid,

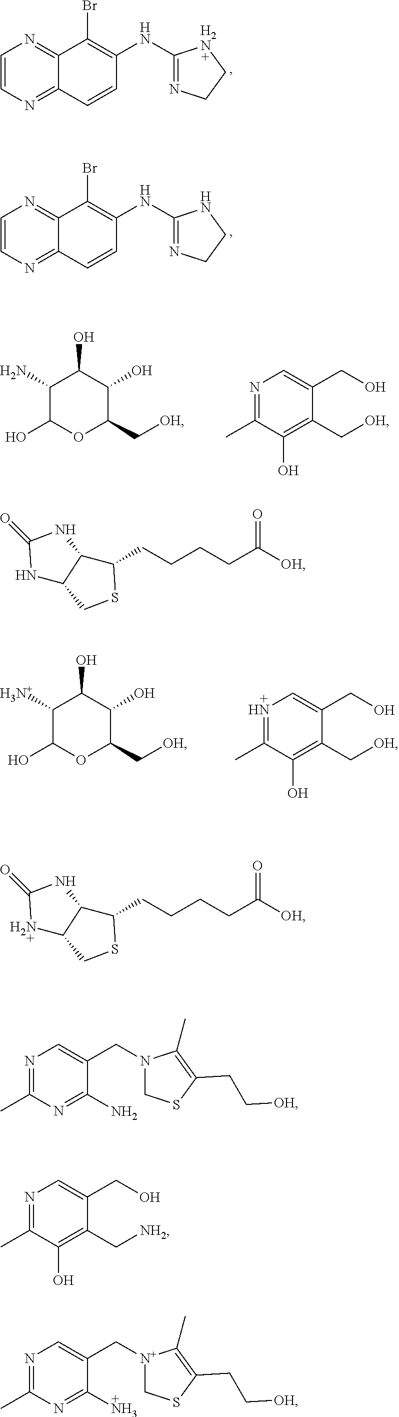

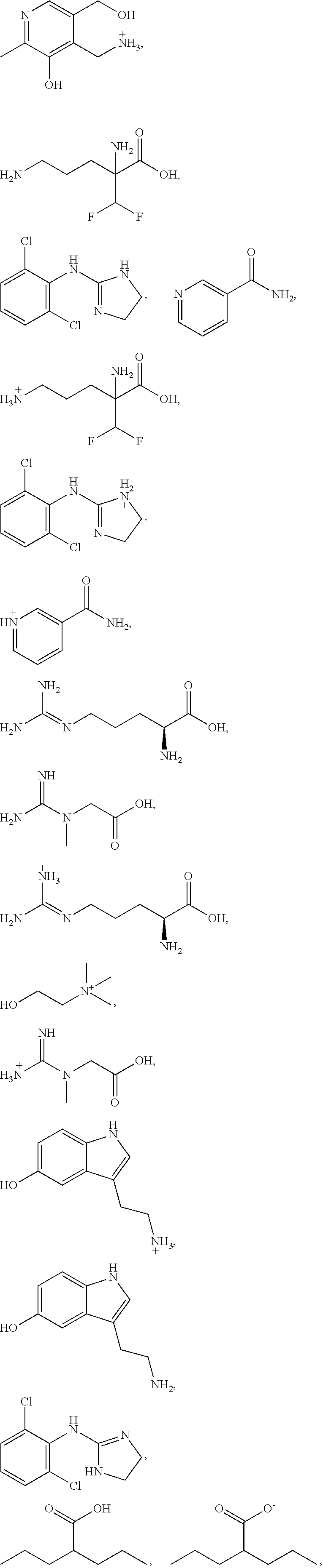

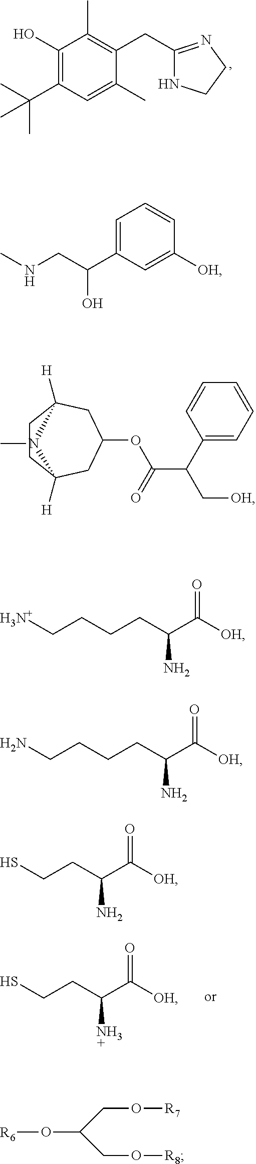

wherein, within the proviso
each R₆, R₇ and R₈ independently represents

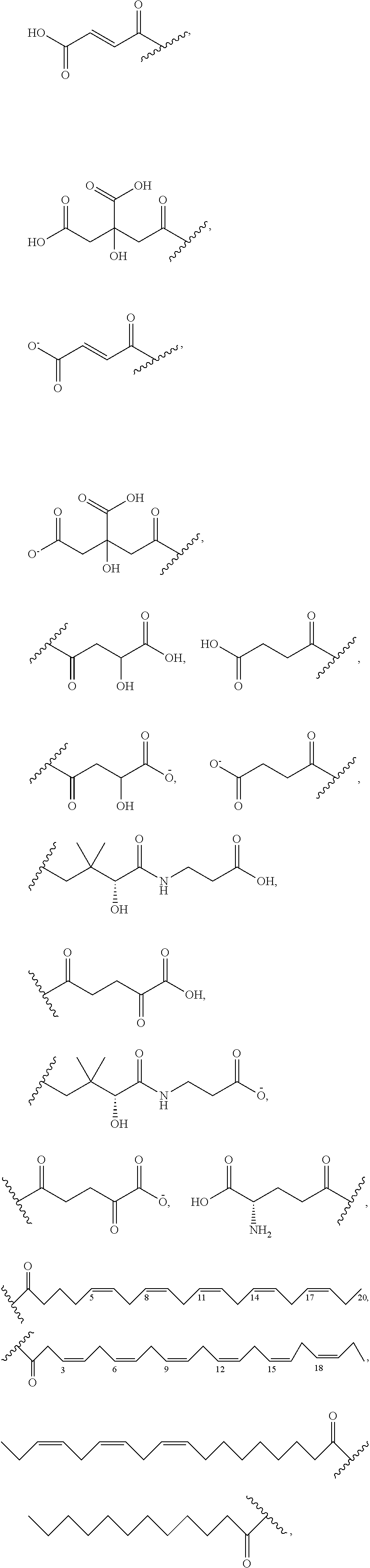

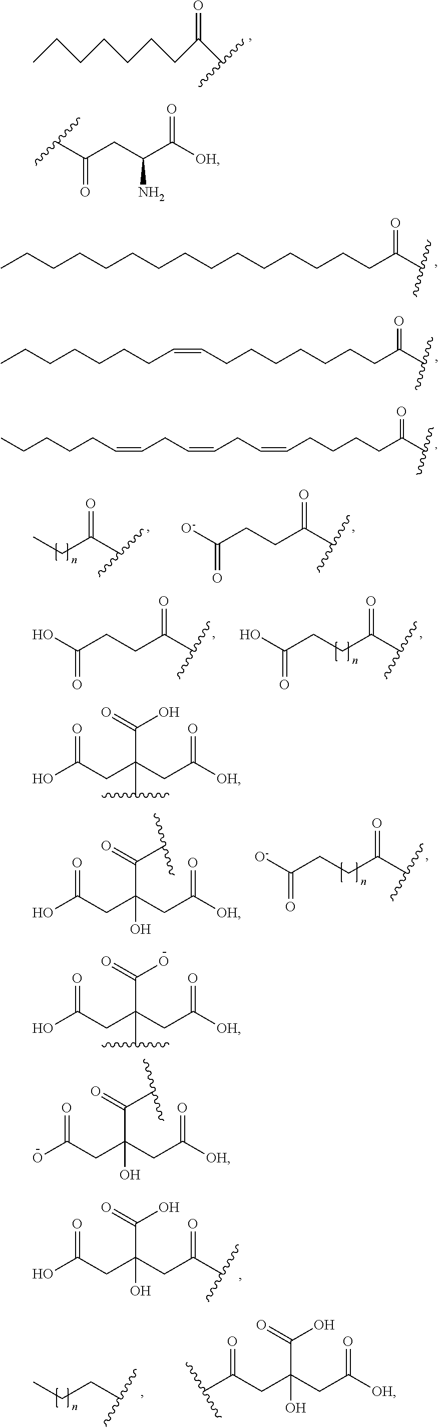

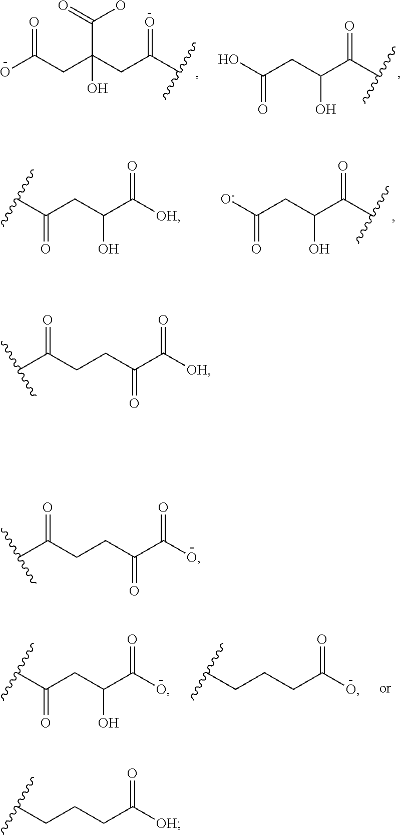

within the proviso;
n=0 to 80.

The compositions are typically compounds in the forms of hydrates or solvates or derivatives or prodrugs of formula I, formula II, formula III, formula IV, formula V, formula VI or formula VII and a moiety [RH] containing compound selected [RH] in which the formula I, formula II, formula III, formula IV, formula V, formula VI or formula VII is protonated/unprotonated based on the derivative functionality and the moiety [RH] of the pharmaceutically acceptable salt is at least in partially ionic form. In some instances, however, for example depending on the pH of the environment, the composition may be in the form of a mixture of formula I, formula II, formula III, formula IV, formula V, formula VI or formula VII and a component represented by [RH]. The invention also provides pharmaceutical compositions comprising compounds of formula I, formula II, formula III, formula IV, formula V, formula VI or formula VII and pharmaceutically acceptable excipients.

The invention herein provides compositions comprising of formula I or pharmaceutical acceptable salts, hydrates, solvates, prodrugs, enantiomers, stereoisomers and derivatives thereof. The invention also provides pharmaceutical compositions comprising compound of formula I or intermediates thereof and one or more of pharmaceutically acceptable excipients and/or pharmaceutically acceptable carriers, including inert vehicles or diluents. These compositions may be used in the treatment of anal and rectal disorder and its associated complications.

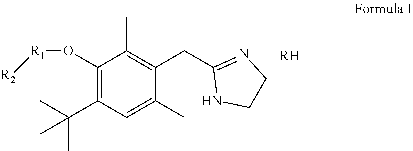

and pharmaceutically acceptable salts, hydrates, solvates, prodrugs, enantiomers, stereoisomers and derivatives thereof;
wherein,
R¹ represents null,

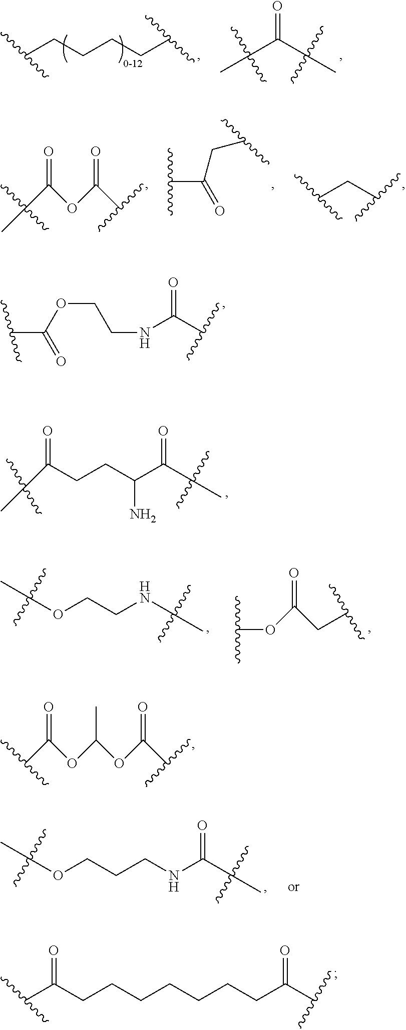

R₂ represents

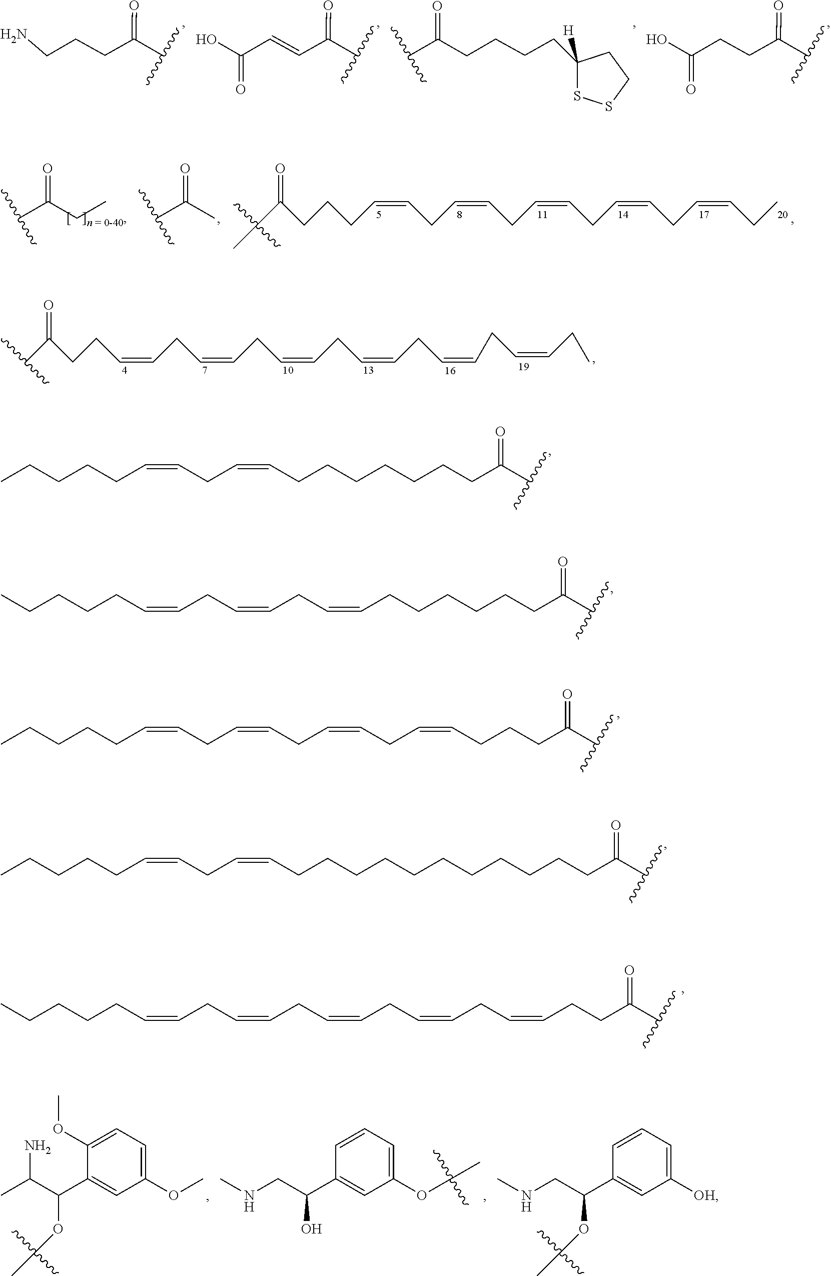

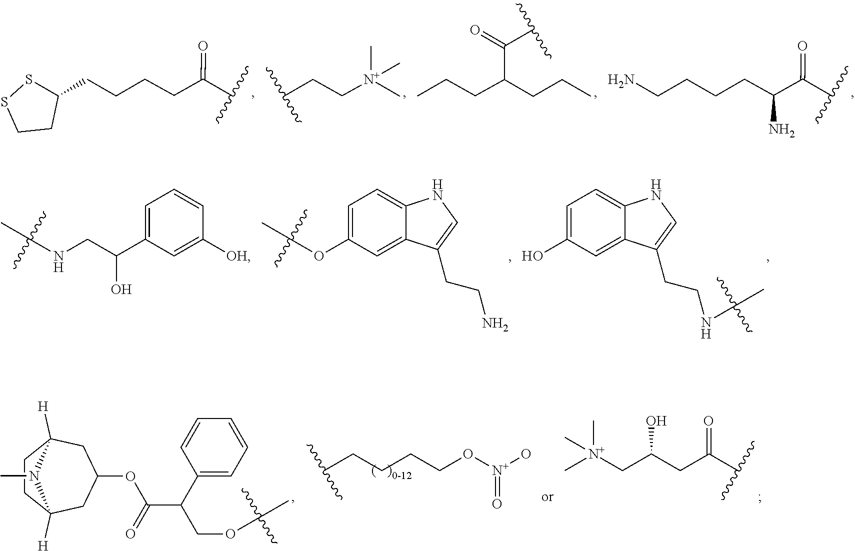

and RH is as defined in Para [0009].

In certain embodiments, the compounds of formula II are described:

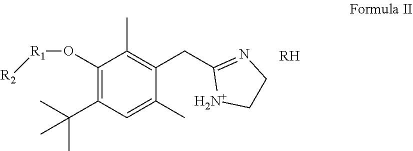

and pharmaceutically acceptable salts, hydrates, solvates, prodrugs, enantiomers, stereoisomers and derivatives thereof;
wherein,
R₁ represents null,

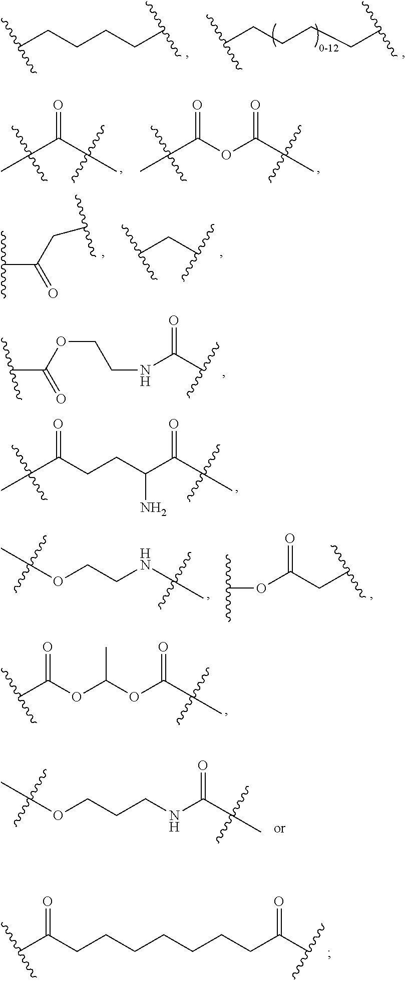

R₂ represents

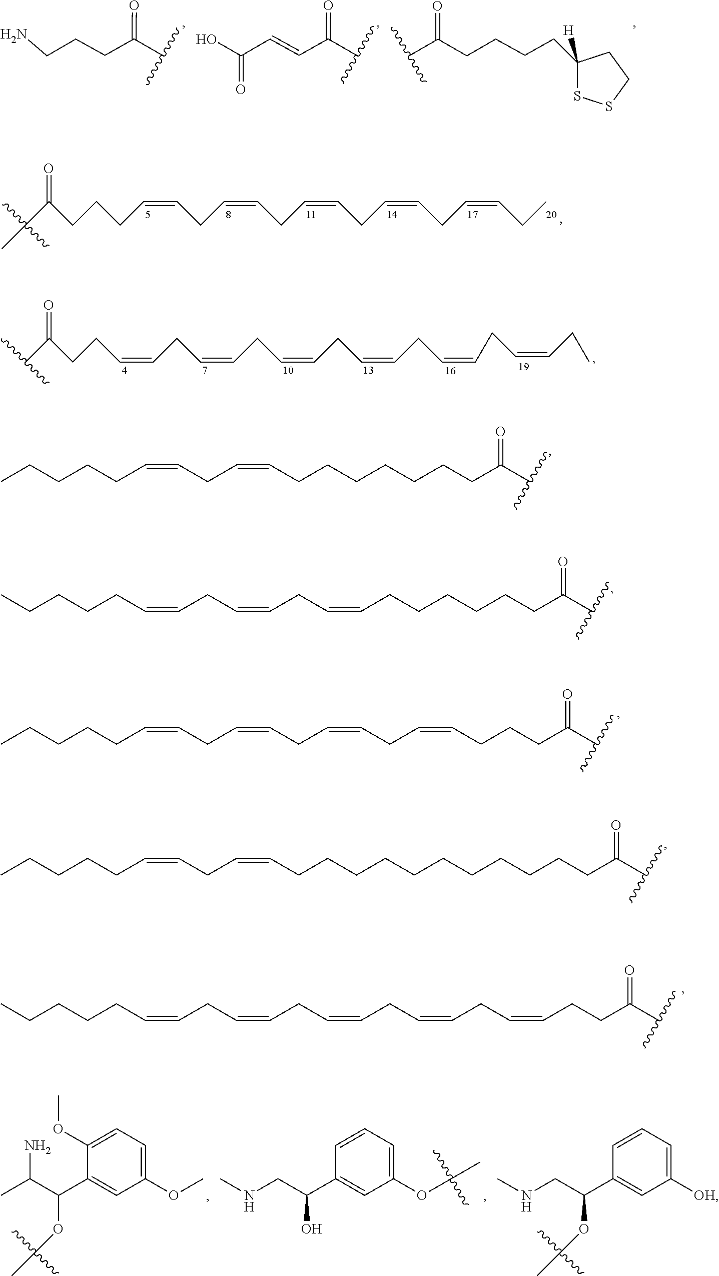

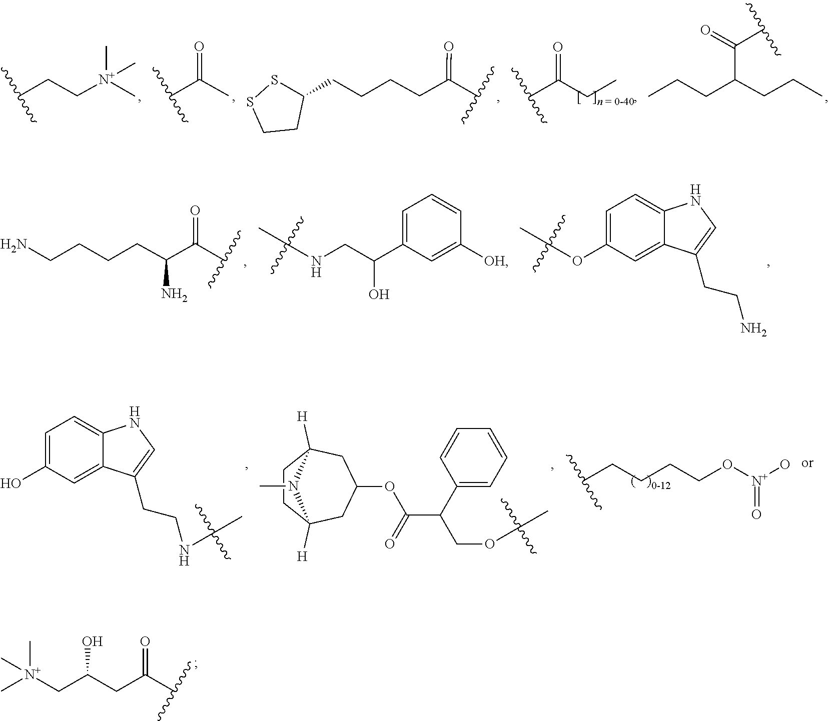

and
RH is as defined in Para [0009].

In certain embodiments, the compounds of formula III are described:

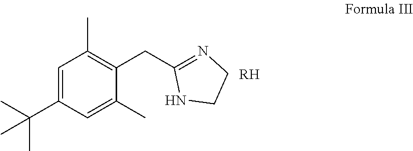

and pharmaceutically acceptable salts, hydrates, solvates, prodrugs, enantiomers, stereoisomers and derivatives thereof;
wherein
RH is as defined in Para [0009].

In certain embodiments, the compounds of formula IV are described:

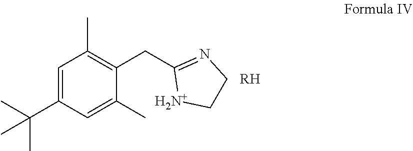

and pharmaceutically acceptable salts, hydrates, solvates, prodrugs, enantiomers, stereoisomers and derivatives thereof;
wherein,
RH is as defined in Para [0009].

In certain embodiments, the compounds of formula V are described:

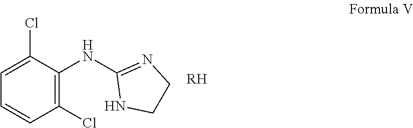

and pharmaceutically acceptable salts, hydrates, solvates, prodrugs, enantiomers, stereoisomers and derivatives thereof;
wherein,
RH is as defined in Para [0009].

In certain embodiments, the compounds of formula VI are described:

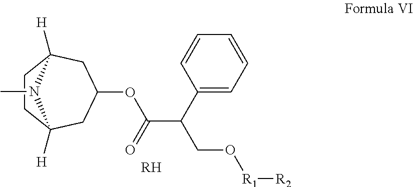

and pharmaceutically acceptable salts, hydrates, solvates, prodrugs, enantiomers, stereoisomers and derivatives thereof;
wherein,
R₁ represents null,

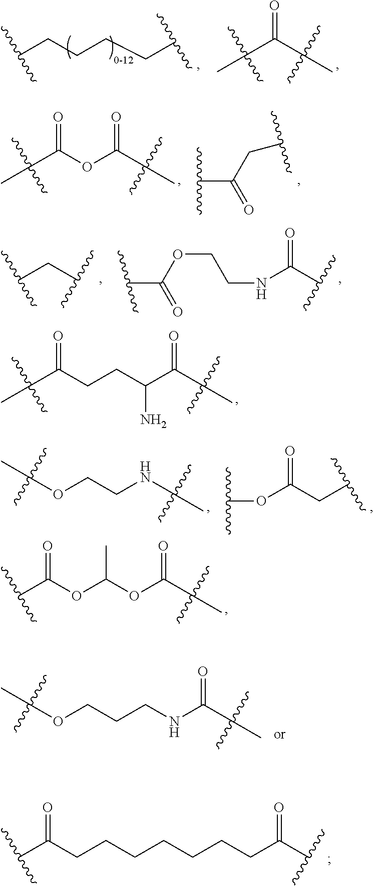

R₂ represents

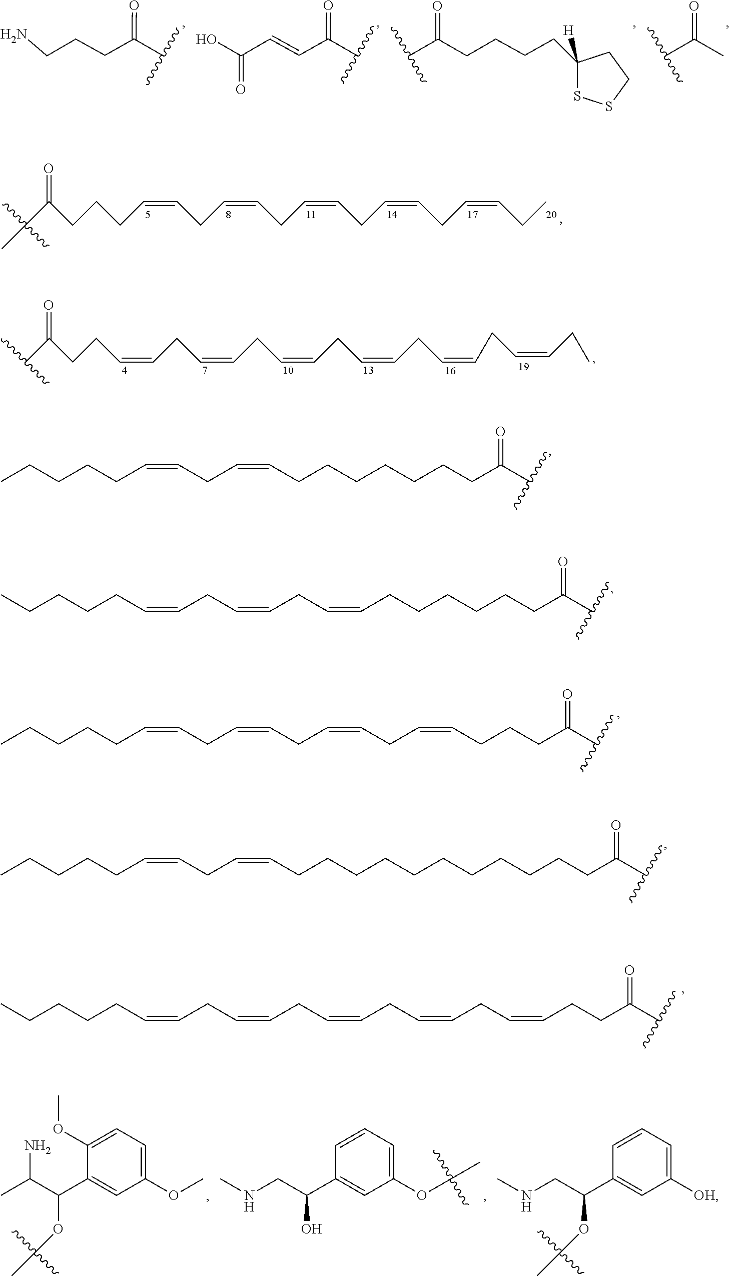

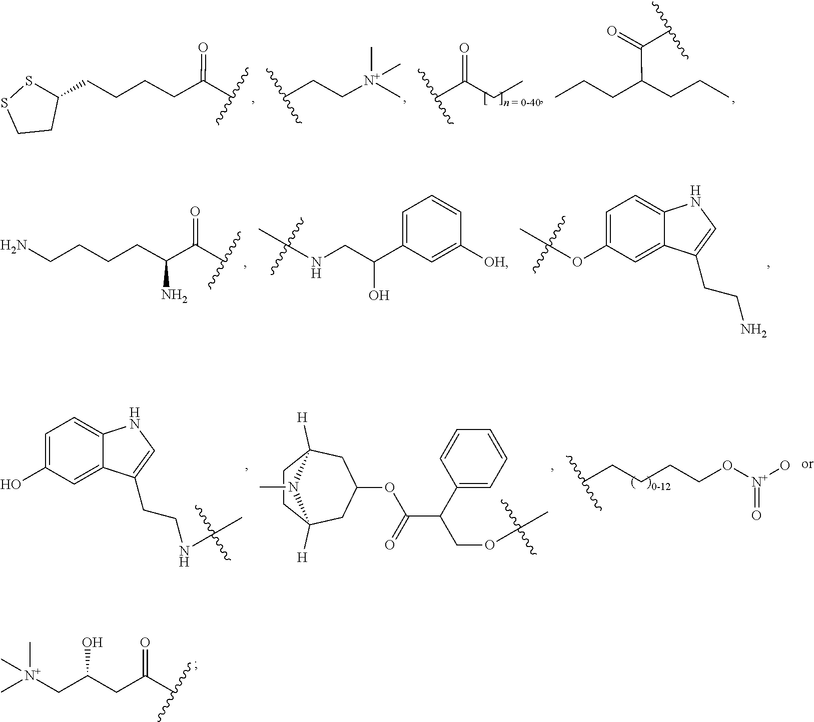

and RH is as define in Para [0009].

In certain embodiments, the compounds of formula VII are described:

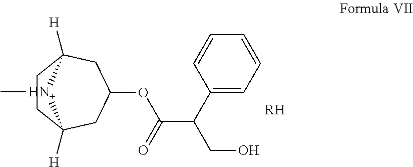

and pharmaceutically acceptable salts, hydrates, solvates, prodrugs, enantiomers, stereoisomers or derivatives thereof;
wherein,
RH is as defined in Para [0009].

The invention also provides pharmaceutical compositions comprises of compounds of formula I, formula II, formula III, formula IV, formula V, formula VI, formula VII, or intermediates, pharmaceutically acceptable salts, hydrates, solvates, prodrugs, enantiomers, stereoisomers or derivative thereof as an active ingredient in a therapeutically effective amount and pharmaceutically acceptable excipients.

The invention also provides pharmaceutical compositions comprising compounds of formula I, formula II, formula III, formula IV, formula V, formula VI, formula VII, or intermediates, pharmaceutically acceptable salts, hydrates, solvates, prodrugs, enantiomers, stereoisomers or derivative and further comprises at least one carrier, diluent, excipient or combination thereof.

The application also discloses a pharmaceutical composition comprising a pharmaceutically acceptable carrier and any of the compounds, mixtures and the compositions herein. The pharmaceutical composition may be formulation depending in the route of administration.

In some aspects, the pharmaceutical compound and composition is formulated for long acting controlled release formulation, sustained release formulation, bioadhesive formulation, mucoadhesive formulation, slow release formulation and modified release formulation or combination thereof.

In some aspects, the pharmaceutical composition is formulated into a solutions, suspension, syrup, emulsion, tablets, capsules, granules/sprinkles controlled release tablets, nasal spray, drops, aerosol creams, suppositories ointments, suppositories, foams, sprays, ointment, emollient, patches, spray, intravenous injections, intramuscular injections, subcutaneous injections, microemulsion, lipid formulation, injectable formulation, transdermal, transmucosal formulation and or transdermal patches and the like and combinations thereof, as would be known to those skilled in the art.

In some aspect the dosage form comprises the pharmaceutical compound and composition and pharmaceutically acceptable suitable carrier.

The compositions described herein have several uses. The present application provides, for example, methods of treating a patient suffering from anal and rectal disorders or its related complications manifested from metabolic or genetic conditions or disorders, gastrointestinal diseases, chronic diseases or disorders; by administering to a subject the compound of formula I, formula II, formula III, formula IV, formula V, formula VI, formula VII or intermediates, derivatives thereof as an active ingredient in a therapeutically effective amount or pharmaceutical composition comprising the same.

Herein the application also provides a kit comprising any of the pharmaceutical compositions disclosed herein. The kit may comprise instructions for use in the treatment of anal and rectal disorders specifically bowel/fecal incontinence, anal pain, anorectal inflammatory diseases, hemorrhoids, anal warts, anal fissures, anorectal abscesses and anal fistulas and its related complications.

In the illustrative embodiments, examples of compounds of formula I, formula II, formula III, formula IV, formula V, formula VI, or formula VII are as set forth below:

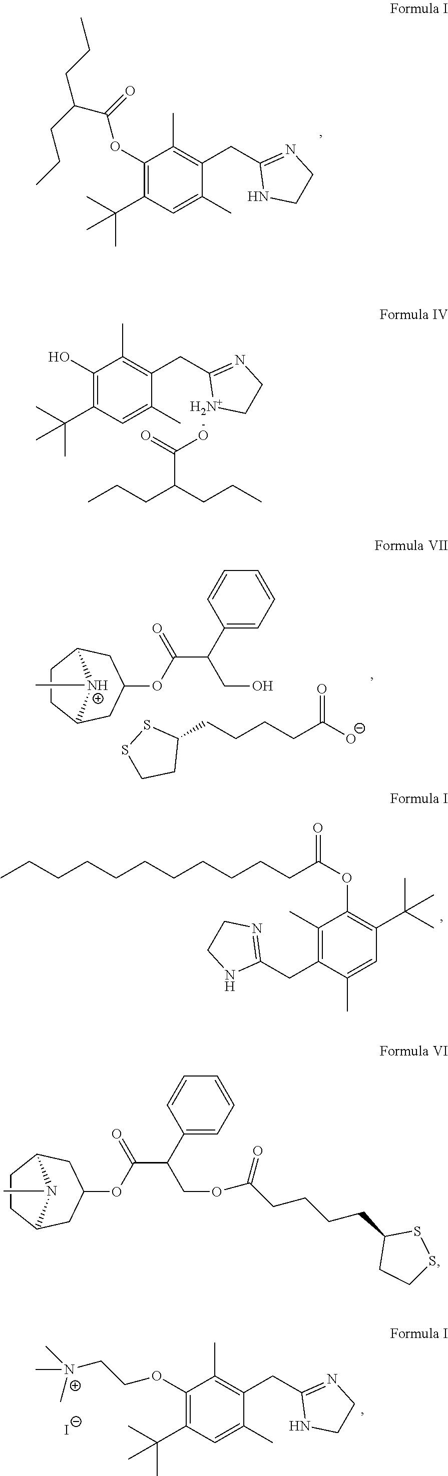

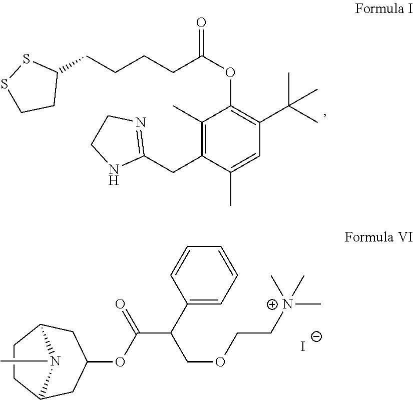

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present disclosure include such modifications and variations and their equivalents.

### DETAILED DESCRIPTION OF THE INVENTION

### Definitions

As used herein, the following terms and phrases shall have the meanings set forth below. Unless defined otherwise, all technical and scientific terms used herein have the same meaning as commonly understood to one of ordinary skill in the art.

As used herein, the following common terms and phrases used in the art such as isomers”, “stereoisomers”, “Diastereomers”, “enantiomers”, “racemic mixture”, “polymorph”, “prodrug”, patient,” “subject,” or “host”, “pharmaceutically acceptable”, “prophylactic or therapeutic”, “predicting”, “treating”, “sustained release”, “parenteral administration” and “administered parenterally”, “systemic administration,” “administered systemically,” “peripheral administration” and “administered peripherally” have the same meaning as understood in the ordinary skill in the art.

The phrase “anal and rectal disorders/anorectal diseases” is an art recognized phrase and includes groups of disorders such as, fecal incontinence, anorectal inflammatory diseases, hemorrhoids, anal warts, anal fissures, anorectal abscesses anorectal pain, and anal fistulas and other related diseases or any other medical condition understood in the art. The term “prodrug” is intended to encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the host animal.

The present disclosure also contemplates prodrugs of the compositions disclosed herein, as well as pharmaceutically acceptable hydrates or solvates of said prodrugs.

The phrase “pharmaceutically acceptable excipients and or carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. Further, the excipients and or carrier used for are non-pyrogenic and non-toxic compatible substances employed in the pharmaceutical formulations and commonly understood to one of ordinary skill in the art.

The pharmaceutical composition may further comprise at least one of a pharmaceutically acceptable excipient selected from a stabilizer, a carrier, a vehicle, a diluent, a surfactant, a filler, a humectant, an adsorbent, an anti-adherent, a binder, a lubricant, a glidant, a super disintegrant, a disintegrant, a preservative, an antioxidant, a solution retarding agent, an absorption accelerator, a wetting agent, an absorbent, a coloring agent, a flavoring agent, a sorbent, a coating agent, a sweetener, a buffering agent, a propellant, or the like and mixtures thereof.

The phrase “therapeutically effective amount” is an art-recognized term. In certain embodiments, the term refers to an amount of a solvate or hydrate or composition disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation.

In many embodiments, the pharmaceutical compositions described herein will incorporate the disclosed compounds or compositions of formula I, formula II, formula III, formula IV, formula V, formula VI or formula VII, to be delivered in an amount sufficient to a patient in a therapeutically effective amount of a compound of formula I, formula II, formula III, formula IV, formula V, formula VI, formula VII, or composition as part of a prophylactic or therapeutic treatment. The desired concentration of compound of formula I, formula II, formula III, formula IV, formula V, formula VI, formula VII, or its pharmaceutical acceptable hydrates or solvates will depend on absorption, inactivation, and excretion rates of the drug as well as the delivery rate of the hydrates or solvates and compositions from the subject compositions. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Additionally, the optimal concentration and/or quantities or amounts of any particular compound of formula I, formula II, formula III, formula IV, formula V, formula VI or formula VII, may be adjusted to accommodate variations in the treatment parameters. Such treatment parameters include the clinical use to which the preparation is put, e.g., the site treated, the type of patient, e.g., human or non-human, adult or child, and the nature of the disease or condition.

The concentration and/or amount of any compound of formula I, formula II, formula III, formula IV, formula V, formula VI or formula VII, may be readily identified by routine screening in animals, e.g., rats, by screening a range of concentration and/or amounts of the material in question using appropriate assays. Known methods are also available to assay local tissue concentrations, diffusion rates of the hydrates or solvates or compositions, and local blood flow before and after administration of therapeutic formulations disclosed herein. When compounds with formula I, formula II, formula III, formula IV, formula V, formula VI or formula VII, such as those disclosed herein are injected adjacent to the loop, released drugs are collected in the dialysate in proportion to their local tissue concentrations. The progress of diffusion of the hydrates or solvates or compositions may be determined thereby with suitable calibration procedures using known concentrations of hydrates or solvates or compositions. In certain embodiments, the dosage of the subject compounds of formula I, formula II, formula III, formula IV, formula V, formula VI or formula VII, provided herein may be determined by reference to the plasma concentrations of the therapeutic composition or other encapsulated materials. For example, the maximum plasma concentration (Cmax) and the area under the plasma concentration-time curve from time 0 to infinity may be used.

The pharmaceutical composition comprising any of the compounds of formula I, formula II, formula III, formula IV, formula V, formula VI or formula VII can be administered by a variety of routes, by way of example and without limitation: parenteral, intravenous, intracoronary, intramuscular intraperitoneal, intra-articular, intra-arterial, intrapleural, intrapericardial, intracardiac, intracavity, intraarterial, intramedullary, intracartilaginous, intradennal, intracapsular, intraorbital, intradermal, intrathecal, intraocular, intraspinal, intrasynovial, intrathoracic, intratracheal, intrauterine, epidural, percutaneous, intravascular, transtracheal, subcuticular, intra-articular, subcapsular, intrastemal, subarachnoid, or subcutaneous injection; inhalation; or oral, topical, nasal, buccal, rectal, ophthalmic, otic, urethral, vaginal, or sublingual administration. Such methods of administration and others contemplated within the scope of the present invention are known to the skilled artisan.

Generally, a composition as described herein may be administered orally; or parenterally, anal or rectally including intravenous, intramuscular, subcutaneous, or intramedullary route. Topical/rectal administration may also be indicated, for example, where the patient is suffering from gastrointestinal disorder that prevent oral administration, or whenever the medication is best applied to the surface of a tissue or organ as determined by the attending physician. Localized administration may also be indicated, for example, when a high dose is desired at the target tissue or organ. For buccal administration the active composition may take the form of tablets or lozenges formulated in a conventional manner.

Further, the compositions may be administered to a subject in need of treatment by controlled release dosage forms (immediate, extended, delayed or pulsed or combination thereof), site specific drug delivery, transdermal drug delivery, patch (active/passive) mediated drug delivery, by stereotactic injection, or in nanoparticles.

This application discloses the pharmaceutical composition comprising the compound of formula I, formula II, formula III, formula IV, formula V, formula VI or formula VII, as an active ingredient in a therapeutically effective amount; and a pharmaceutically acceptable excipient, wherein the pharmaceutical composition is formulated into different dosage forms.

This application discloses the pharmaceutical composition formulated in dosage form selected from the group consisting of tablet, caplet, gel, gel cap, enema, sublingual tablet, flash, mucoadhesive tablet/patches, multilayer tablets, capsules, capsules containing tablet, injectable, i.v., depot injection, aerosol, concentrate, dressing, syrup, film, granule, controlled-release form, sustained-release form, suppository, tampons, pessaries, pills, jelly, form, paste, pastille, pellet, spray, troche, douche, inhalant, lozenge, powders, beads, granules, nanoparticles, oral spray, oral solution or suspension, nasal spray, mucoadhesive spray, intra nasal spray, nasal inhaler, liquid solution, elixirs, emulsions, microemulsions, suppositories subdermal autoinjector, intramuscular autoinjector, injection, stereotactic injection, liquid suspension, intravenous suspension, sterile parenteral solution, sterile parenteral suspension, sterile non-parenteral solution, sterile non-parenteral suspension, topical ointment, topical paste, topical cream, topical lotion, topical gel, transdermal patch, dermal patch, implants, intrauterine device and know dosage forms containing suitable quantities of the compounds and or composition disclosed in the instant invention. For oral administration either solid or liquid unit dosage forms can be prepared. The methods for the preparation of the dosage forms contemplated herein are described in standard pharmaceutical science, the disclosures of which are hereby incorporated herein in their entirety. Any ingredients used in the present formulation should not degrade or decompose a significant portion of the compounds disclosed here prior to administration.

In an embodiment, the present disclosure also discloses a method of the treatment, prevention or amelioration of a anorectal diseases, fecal incontinence, hemorrhoids, anal warts, anal fissures, anorectal abscesses and anal fistulas or an associated complication, wherein the method comprises of administering to a subject the compound of formula I, formula II, formula III, formula IV, formula V, formula VI or formula VII, as an active ingredient in a therapeutically effective amount, or the pharmaceutical composition comprising the same

Generally, in carrying out the methods detailed in this application, an effective dosage for the compounds of formula I, formula II, formula III, formula IV, formula V, formula VI or formula VII, is in the range of about 0.01 mg/kg/day to about 100 mg/kg/day in single or divided doses, for instance 0.01 mg/kg/day to about 50 mg/kg/day in single or divided doses. The compounds of formula I, formula II, formula III, formula IV, formula V, formula VI or formula VII, may be administered to a subject at a dose of, for example, less than 0.2 mg/kg/day, 0.5 mg/kg/day, 1.0 mg/kg/day, 5 mg/kg/day, 10 mg/kg/day, 20 mg/kg/day, 30 mg/kg/day, or 40 mg/kg/day. Compounds of formula I, formula II, formula III, formula IV, formula V, formula VI or formula VII, may also be administered to a human patient at a dose of, for example, between 0.1 mg and 1000 mg, between 5 mg and 80 mg, or less than 1.0, 9.0, 12.0, 20.0, 50.0, 75.0, 100, 300, 400, 500, 800, 1000, 2000, 5000 mg per day. In certain embodiments, the compositions herein are administered at an amount that is less than 95%, 90%, 80%, 70%, 60%, 50%, 40%, 30%, 20%, or 10% of the compound of formula I, formula II, formula III, formula IV, formula V, formula VI or formula VII, required for the same therapeutic benefit.

An effective amount of the compound of formula I, formula II, formula III, formula IV, formula V, formula VI or formula VII, described herein refers to the amount of one of said hydrates or solvates or compositions which is capable of inhibiting or preventing a disease.

The compositions may be administered alone or in combination with pharmaceutically acceptable carriers, vehicles or diluents, in either single or multiple doses. The compounds of formula I, formula II, formula III, formula IV, formula V, formula VI or formula VII, may also comprises polymers, hydrogel and or liposomes and others well known in the pharmaceutical art.

In another embodiment, tablets, may comprise e.g. 1 to 100, 50 to 250, 150 to 500 mg, or 350 to 800 mg e.g. 10, 50, 100, 300, 500, 700, 800 mg of the compounds of formula I, formula II, formula III, formula IV, formula V, formula VI or formula VII, disclosed herein, for instance, compounds of formula I, formula II, formula III, formula IV, formula V, formula VI, formula VII or pharmaceutical acceptable hydrates or solvates, salts or derivatives of a compounds of formula I, formula II, formula III, formula IV, formula V, formula VI or formula VII.

The dosage administered will be dependent upon the intensity of the fecal incontinence, anorectal disorders, hemorrhoids, anal warts, anal fissures, anorectal abscesses and anal fistulas; the type of host involved, including its age, health and weight; the kind of concurrent treatment, if any; the frequency of treatment and therapeutic ratio.

Illustratively, dosage levels of the administered active ingredients are: intravenous, 0.1 to about 200 mg/kg; intramuscular, 1 to about 500 mg/kg; orally, 5 to about 1000 mg/kg; intranasal instillation, 5 to about 1000 mg/kg; and aerosol, 5 to about 1000 mg/kg of subject body weight.

Expressed in terms of concentration, an active ingredient of the present invention can be presented in the pharmaceutical compositions for localized use about the cutis, intranasally, pharyngolaryngeally, bronchially, intravaginally, rectally, or ocularly in a concentration of from about 0.01 to about 50% w/w of the composition; preferably about 1 to about 20% w/w of the composition; and for parenteral use in a concentration of from about 0.05 to about 50% w/v of the composition and preferably from about 5 to about 20% w/v.

In one embodiment, the first portion contains the first pharmaceutically active agent and the second portion contains the second pharmaceutically active agent. In one embodiment, one of the portions contains a third pharmaceutically active agent. In one embodiment one of the portions contains a second immediate release portion of the same pharmaceutically active agent as that contained in the tablet core.

Each dosage form contains a therapeutically effective amount of active agent. In one embodiment of dosage forms that mimic a twice daily dosing profile, approximately 30 wt. % to 70 wt. %, preferably 40 wt. % to 60 wt. %, of the total amount of active agent in the dosage form is released in the initial pulse, and, correspondingly approximately 70 wt. % to 3.0 wt. %, preferably 60 wt. % to 40 wt. %, of the total amount of active agent in the dosage form is released in the second pulse. For dosage forms mimicking the twice daily dosing profile, the second pulse is preferably released approximately 3 hours to less than 14 hours, and more preferably approximately 5 hours to 12 hours, following administration.

Another dosage form contains a compressed tablet or a capsule having a drug-containing immediate release dosage unit, a delayed release dosage unit and an optional second delayed release dosage unit. In this dosage form, the immediate release dosage unit contains a plurality of beads, granules particles that release drug substantially immediately following oral administration to provide an initial dose. The delayed release dosage unit contains a plurality of coated beads or granules, which release drug approximately 3 hours to 14 hours following oral administration to provide a second dose.

For purposes of transdermal (e.g., topical) administration, dilute sterile, aqueous or partially aqueous solutions (usually in about 0.1% to 5% concentration), otherwise similar to the above parenteral solutions, may be prepared.

Methods of preparing various pharmaceutical compositions with a certain amount of one or more compounds of formula I, formula II, formula III, formula IV, formula V, formula VI or formula VII or other active agents are known, or will be apparent in light of this disclosure, to those skilled in this art. For examples of methods of preparing pharmaceutical compositions, see Remington's Pharmaceutical Sciences, Mack Publishing Company, Easton, Pa., 19th Edition (1995).

In addition, in certain embodiments, subject compositions of the present application maybe lyophilized or subjected to another appropriate drying technique such as spray drying. The subject compositions may be administered once, or may be divided into a number of smaller doses to be administered at varying intervals of time, depending in part on the release rate of the compositions and the desired dosage.

Methods of preparing these formulations or compositions include the step of bringing into association subject compositions that is a certain amount of compound of formula I, formula II, formula III, formula IV, formula V, formula VI or formula VII with a pharmaceutically acceptable carrier and, optionally, one or more accessory ingredients. In general, the formulations are prepared by uniformly and intimately bringing into association a subject composition with liquid carriers, or finely divided solid carriers, or both, and then, if necessary, shaping the product.

The compounds of formula I, formula II, formula III, formula IV, formula V, formula VI or formula VII, described herein or the pharmaceutical compositions comprising the same may be formulated and administered in the form of an inhalant or aerosol formulations. The inhalant or aerosol formulations may comprise one or more agents, such as adjuvants, diagnostic agents, imaging agents, or therapeutic agents useful in inhalation therapy. The final aerosol formulation may for example contain 0.005-90% w/w, for instance 0.005-50%, 0.005-5% w/w, or 0.01-1.0% w/w, of medicament relative to the total weight of the formulation.

Formulations for rectal or vaginal administration may be presented as a suppository, which may be prepared by mixing a subject composition with one or more suitable non-irritating carriers comprising, for example, cocoa butter, polyethylene glycol, a suppository wax, or a salicylate, and which is solid at room temperature, but liquid at body temperature and, therefore, will melt in the appropriate body cavity and release the encapsulated compound(s) and composition(s). Formulations which are suitable for vaginal administration also include pessaries, tampons, creams, gels, pastes, foams, or spray formulations containing such carriers as are known in the art to be appropriate.

Also disclosed is a kit comprising the compound of formula I, formula II, formula III, formula IV, formula V, formula VI or formula VII, as an active ingredient in a therapeutically effective amount, or a pharmaceutical composition as described herein, and an instruction for use in the treatment of anorectal diseases and associated its complications thereof.

### METHODS OF SYNTHESIS

### Example 1: CLX-SYN-G162D-C01

### IUPAC Name of CLX-SYN-G162D-C01: 6-(tert-butyl)-3-((4,5-dihydro-1H-imidazol-2-yl)methyl)-2,4-dimethylphenyl dodecanoate

### Chemical Structure of CLX-SYN-G162D-C01

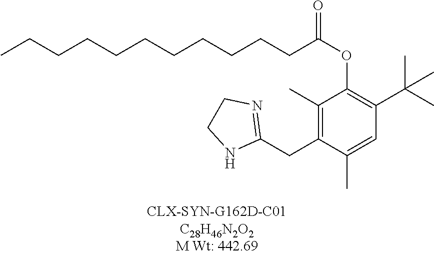 [SMILES: CCCCCCCCCCCC(=O)Oc1c(C(C)(C)C)cc(C)c(CC2=NCCN2)c1C]

### Synthesis of CLX-SYN-G162D-C01

The synthesis of CLX-SYN-G162D-C01 is a four step process which starts from oxymetazoline hydrochloride. First step is the free basing of the oxymetazoline hydrochloride using aqueous ammonia solution. The second stage is the benzyl protection of Oxymetazoline with benzyl chloride. Esterification of phenol with lauoryl chloride followed by benzyl deprotection using Pd/C in methanol generates CLX-SYN-G162D-C01 as shown in Scheme 1. Detailed experimental procedures for all the stages is provided in the below sections.

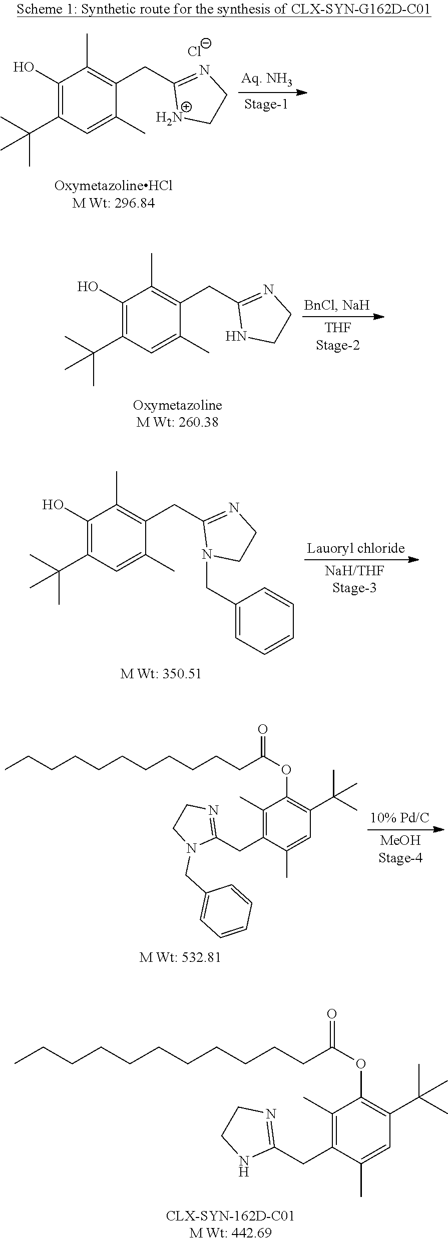

### Synthesis of Stage-1 of CLX-SYN-G162D-C01

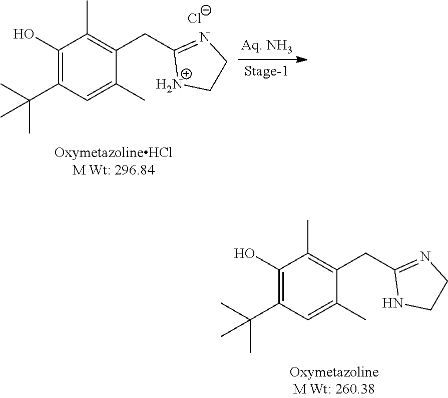 [SMILES: Cc1cc(C(C)(C)C)c(O)c(C)c1CC1=NCC[NH2+]1 ; [Cl-] ; Cc1cc(C(C)(C)C)c(O)c(C)c1CC1=NCCN1]

### Experimental Procedure

To a stirred solution of oxymetazoline hydrochloride (1.0 eq.) in water (20.0 vol.), aq. ammonia solution (1.0 vol.) was added at 25-30° C. and the reaction mass was stirred for 1-2 h at 25-30° C. The solid precipitated was filtered, washed with water (5 vol.) and suck dried for 1-2 h. The wet compound was dried under vacuum at 60-65° C. for about 16 h to afford oxymetazoline as off white solid.

### Synthesis of Stage-2 of CLX-SYN-G162D-C01:

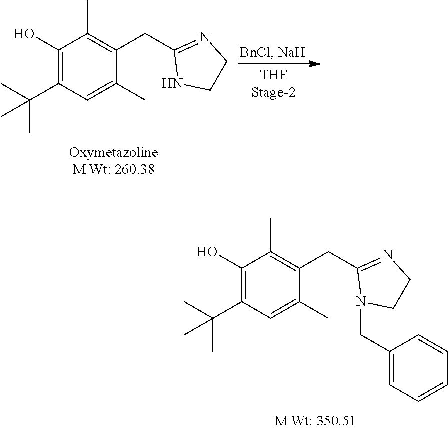 [SMILES: Cc1cc(C(C)(C)C)c(O)c(C)c1CC1=NCCN1 ; Cc1cc(C(C)(C)C)c(O)c(C)c1CC1=NCCN1Cc1ccccc1]

### Experimental Procedure

To a stirred solution of St-1 (1 eq.) in DMF (14.0 vol.), Benzyl chloride (1.05 eq.) was added and cooled to 0-5° C. To the reaction mass (RM), NaH (1.05 eq.) was added portion wise (in three lots) at 0-5° C. and stirred for 2 h at the same temperature. Quenched the reaction with water (20 vol.), extracted with Ethyl acetate (EtOAc) (20 vol.). Organic layer was washed with water (2×10 vol.) and brine solution (10 vol.); concentrated under reduced pressure and product was isolated by adding n-heptane. The isolated solid was filtered, dried under vacuum to afford the required compound as cream color solid.

### Synthesis of Stage-3 of CLX-SYN-G162D-C01:

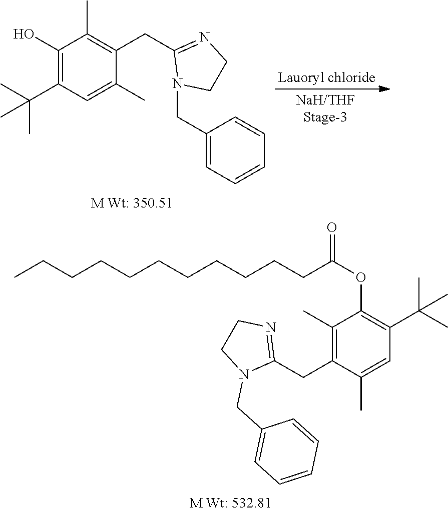 [SMILES: Cc1cc(C(C)(C)C)c(O)c(C)c1CC1=NCCN1Cc1ccccc1 ; CCCCCCCCCCCC(=O)Oc1c(C(C)(C)C)cc(C)c(CC2=NCCN2Cc2ccccc2)c1C]

### Experimental Procedure

To a stirred solution of St-2 (1 eq.) in Dry tetrahydrofuran (THF) (14.0 vol.), NaH (1.5 eq.) was added portion wise at 0-5° C. and stirred for 10-15 min. lauryl chloride (1.1 eq.) at 0-5° C. was added and stirred for 2 h at the same temperature. Quenched the reaction with water (20 vol.), extracted with EtOAc (20 vol.). Organic layer was washed with water (2×10 vol.) and brine solution (10 vol.). Organic layer was concentrated and product isolated in n-heptane. The solid was filtered, and dried under vacuum to afford as required compound as pale yellow color solid.

### Synthesis of Stage-4 of CLX-SYN-G162D-C01:

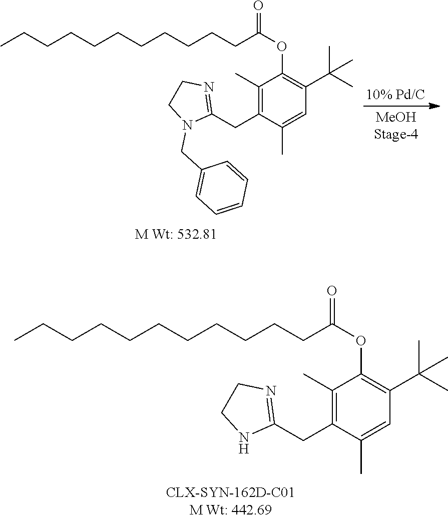

### Experimental Procedure

To a stirred solution of St-3 (1 eq.) in methanol (10.0 vol.), 10% Pd/C was added at 25-30° C. and reaction materials, stage 3 material is stirred for 12.0 h at 25-30° C. under H2 atmosphere. Reaction materials filtered over celite, concentrated and the crude compound was purified by Column Chromatography on neutral alumina using EtOAc/Hexanes as eluents to result in the required compound as pale yellow color liquid.

Details of all the Stage-4 executed is given below in the Table 1.

TABLE 1
Details of the Stage-4 reactions

| | | | | Purity by |
|---|---|---|---|---|
| | Input | Output | Yield | HPLC |
| S. No. | (g) | (g) | (%) | (% area) |
| 1 | 0.2 | 40 mg | 24 | NA |
| 2 | 1.0 | 175 mg | 21 | 96.13 |

### Structure Characterization Data of CLX-SYN-G162D-C01

Chemical structure of CLX-SYN-G162D-C01 has been established using various spectroscopic techniques such as IR, NMR, Mass and 13C NMR and results are summarized below.

Infrared Spectrophotometry: The FT-IR spectrum of CLX-SYN-G162D-C01 recorded using Sodium chloride cells in FT-IR spectrophotometer and exhibits following IR absorption bands characteristic of its chemical structure as tabulated below.

IR (cm⁻¹): Characteristic Absorption Bands:

| Position of the Functional group | Wave number (cm⁻¹) |
|---|---|
| —NH | 3018 |
| Alkyl C—H | 2859-2926 |
| —C═N | 2402 |
| —C═O | 1661 |
| C═C (Aromatic) | 1522 |
| ═C—O | 1216 |

### ¹H NMR (400 MHz, DMSO-D6) of CLX-SYN-G162D-C01:

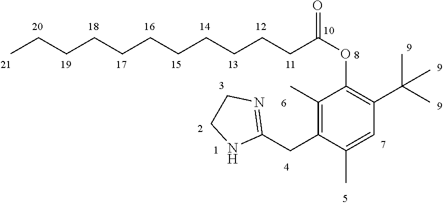 [SMILES: CCCCCCCCCCCC(=O)Oc1c(C(C)(C)C)cc(C)c(CC2=NCCN2)c1C]

### Assignment of Chemical Shifts of Protons

| Position of the proton | Chemical shift, ppm |
|---|---|
| 21 (3H) | 0.85 (3H, t) |
| 12-19 (16H) | 1.20-1.30 (16H, m) |
| 9 (9H) | 1.33 (9H, s) |
| 20 (2H) | 1.50-1.55 (2H, m) |
| 5 (3H) | 1.98 (3H, s) |
| 6 (3H) | 2.05 (3H, s) |
| 11 (2H) | 2.41 (2H, t) |
| 2 (2H) | 3.54 (2H, t) |
| 3 (2H) | 3.83 (2H, t) |
| 4 (2H) | 3.92 (2H, s) |
| 10 (2H) | 4.76 (2H, s) |
| 7 (1H) | 6.76 (1H, s) |
| 1 (NH) | 7.64 (1H, s) |

¹³C NMR (100 MHz, DMSO-d₆): Assignment of Chemical Shifts to Carbon Atoms

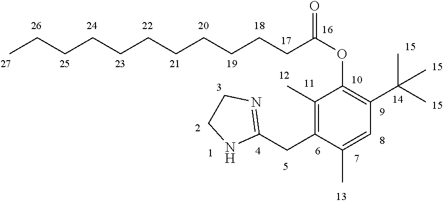 [SMILES: CCCCCCCCCCCC(=O)Oc1c(C(C)(C)C)cc(C)c(CC2=NCCN2)c1C]

| Position of the | |
|---|---|
| Carbon atoms | Chemical shift, ppm |
| C-12 | 13.02 |
| C-13 | 13.94 |
| C-27 | 19.79 |
| C-26 | 22.09 |
| C-18 | 23.69 |
| C-19 | 28.57 |
| C-20 | 28.71 |
| C-21 | 28.92 |
| C-22 | 28.96 |
| C-23 | 29.00 |
| C-24 | 29.02 |
| C-15 (3 x C) | 29.81 |
| C-25 | 31.29 |
| C-14 | 32.54 |
| C-17 | 34.08 |
| C-5 | 35.91 |
| C-2 | 46.08 |
| C-3 | 52.30 |
| C-7 | 124.74 |
| C-11 | 124.89 |
| C-8 | 126.65 |
| C-9 | 132.79 |
| C-6 | 134.69 |
| C-10 | 151.00 |
| C-4 | 158.88 |
| C-16 | 175.64 |

MS: 443.6 (+ve mode).

### Example 1A: Synthesis of CLX-SYN-G162D-CO1

### Route of Synthesis

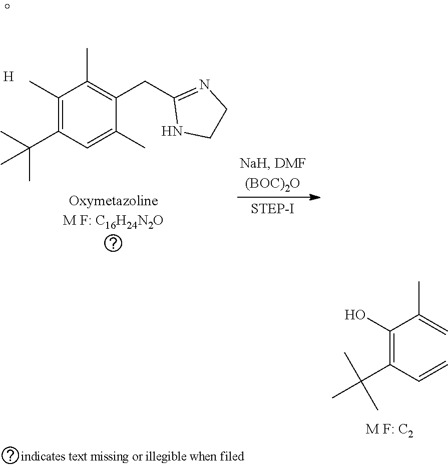

### Procedure:

### Step-I:

Sodium hydride (1.1 Eq) added to a solution of oxymetazoline (1.0 Eq), Di-tert-butyl bicarbonate (1.0 Eq) and Dimethyl formamide (10 mL) at 0-5° C. over a period of 5-10 min. Mass stirred for 40-60 min. During the addition of ice water at 0-5° C. solid was separated, Filter the solid. Solid compound dissolved in ethyl acetate. Organic layer dried over sodium sulphate and filtered, and concentrate under vacuum to get solid.

### Step-II

Sodium hydride (1.5 Eq) added to a solution of Step-I (1.0 Eq) and Dimethyl formamide (10 mL) at 0-5° C. over a period of 5-10 min. Mass stirred for 30-40 min. Slowly valprioc acid chloride (1.5 Eq) was added to above mass at 0-5° C. and maintained for 40-60 min. Ice water was added to reaction mass at 0-5° C., extracted with ethyl acetate (50 mL×2 times) and Organic layer was dried over sodium sulphate and filtered, concentrated under vacuum to get oily mass. This compound purified by column chromatography by using mixture of Ethyl acetate and hexane and volatile was concentrated to get solid.

### Step-III

Ethyl acetate HCl solution was added to a solution of Step-II in ethyl acetate (1 mL) at 20-25° C. over a period of 2-5 min. Mass stirred for 3-5 hours and concentrated under vacuum to get oily mass. This compound purified by column chromatography by using mixture of Ethyl acetate and hexane and volatile was concentrated to get solid.

### Example 2: CLX-SYN-G162D-C02

### IUPAC Name of CLX-SYN-G162D-C02: 6-(tert-butyl)-3-((4,5-dihydro-1H-imidazol-2-yl)methyl)-2,4-dimethylphenyl (R)-5-(1,2-dithiolan-3-yl)pentanoate

### Chemical Structure of CLX-SYN-G162D-C02:

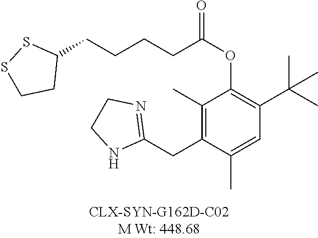

### Synthesis of CLX-SYN-G162D-C02

The proposed synthetic route of CLX-SYN-G162D-C02 is a four step process which starts from oxymetazoline hydrochloride (Scheme 1).

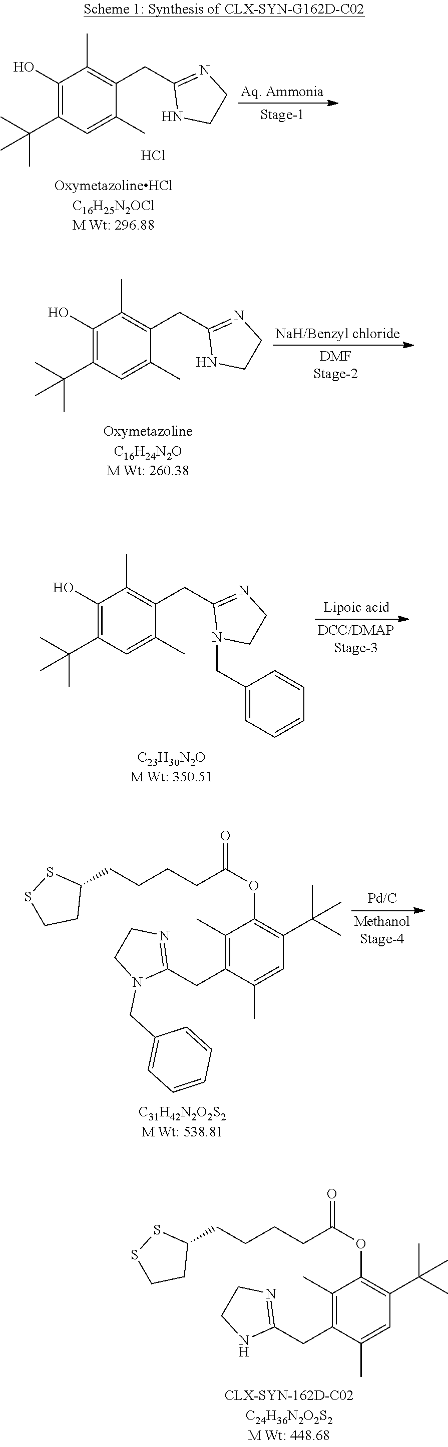 [SMILES: Cc1cc(C(C)(C)C)c(O)c(C)c1CC1=NCCN1 ; Cl ; Cc1cc(C(C)(C)C)c(O)c(C)c1CC1=NCCN1 ; Cc1cc(C(C)(C)C)c(O)c(C)c1CC1=NCCN1Cc1ccccc1 ; Cc1cc(C(C)(C)C)c(OC(=O)CCCC[C@@H]2CCSS2)c(C)c1CC1=NCCN1Cc1ccccc1 ; Cc1cc(C(C)(C)C)c(OC(=O)CCCC[C@@H]2CCSS2)c(C)c1CC1=NCCN1]

First step is the free basing of the oxymetazoline hydrochloride using aqueous ammonia solution. The second stage is the benzyl protection of oxymetazoline with benzyl chloride. Esterification of phenol of Stage-2 with lipoic acid (i.e. Stage-3) followed by benzyl deprotection using Pd/C in methanol (i.e. Stage-4) generates CLX-SYN-G162D-C02 as shown in Scheme 1. Detailed experimental procedures for all the stages is provided in the below sections.

### Synthesis of Stage-1 of CLX-SYN-G162D-C02

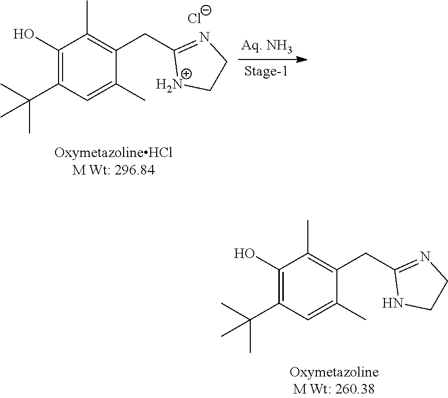 [SMILES: Cc1cc(C(C)(C)C)c(O)c(C)c1CC1=NCC[NH2+]1 ; [Cl-] ; Cc1cc(C(C)(C)C)c(O)c(C)c1CC1=NCCN1]

### Experimental Procedure

To a stirred solution of oxymetazoline hydrochloride (1.0 eq.) in water (20.0 vol.), aq. ammonia solution (1.0 vol.) was added at 25-30° C. and the reaction mass was stirred for 1-2 h at 25-30° C. The solid precipitated was filtered, washed with water (5 vol.) and suck dried for 1-2 h. The wet compound was dried under vacuum at 60-65° C. for about 16 h to afford oxymetazoline as off white solid.

### Synthesis of Stage-2 of CLX-SYN-G162D-C02:

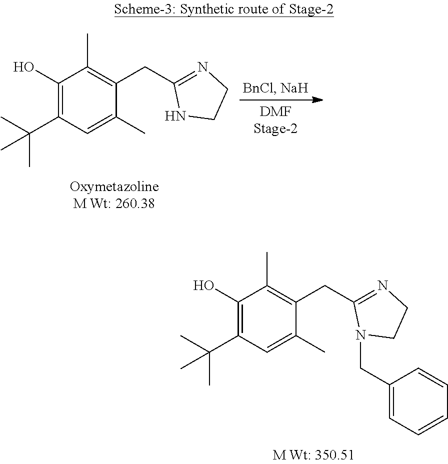 [SMILES: Cc1cc(C(C)(C)C)c(O)c(C)c1CC1=NCCN1 ; Cc1cc(C(C)(C)C)c(O)c(C)c1CC1=NCCN1Cc1ccccc1]

### Experimental Procedure

To a stirred solution of Stage-1 (1 eq.) in DMF (14.0 vol.), Benzyl chloride (1.05 eq.) was added and cooled to 0-5° C. To the RM, NaH (1.05 eq.) was added portion wise (in three lots) at 0-5° C. and stirred for 2 h at the same temperature. Quenched the reaction with water (20 vol.), extracted with EtOAc (20 vol.). Organic layer was washed with water (2×10 vol.) and brine solution (10 vol.); concentrated under reduced pressure and product was isolated by adding n-heptane. The isolated solid was filtered, dried under vacuum to afford the required compound as cream color solid.

### Synthesis of Stage-3 of CLX-SYN-G162D-C02:

### Using Lipoic Acid Chloride Route:

The synthesis of the Stage-3 compound was initially attempted following the acid chloride route as shown in Scheme-4. The lipoic acid chloride is generated by treating lipoic acid with thionyl chloride.

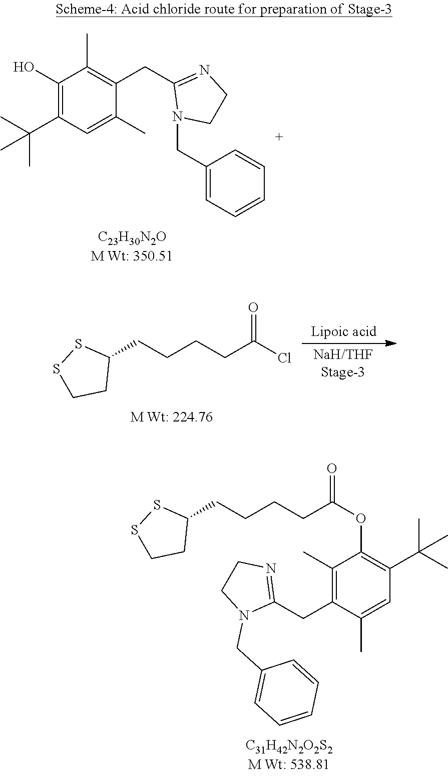 [SMILES: Cc1cc(C(C)(C)C)c(O)c(C)c1CC1=NCCN1Cc1ccccc1 ; O=C(Cl)CCCC[C@@H]1CCSS1 ; Cc1cc(C(C)(C)C)c(OC(=O)CCCC[C@@H]2CCSS2)c(C)c1CC1=NCCN1Cc1ccccc1]

The acid chloride preparation was attempted using oxalyl chloride in DCM as solvent. After completion the solvent was evaporated and the crude material was used directly in the next reaction.

### Synthesis Via Amide Coupling:

As the acid chloride route didn't result in the required product formation, further attempts were focused on the coupling reaction of the lipoic acid with the Stage-2 amine. Towards this end the initial reaction was attempted by treating the Stage-II with lipoic acid in presence of DCC/DMAP in DCM as solvent. After completion of the reaction followed by work up, the crude compound was purified by silica gel column chromatography using hexane/EtOAc as eluents resulting in the required product in about 35% yield.

For the synthesis of Stage-3, the coupling reaction in presence of DCC generates the required product and hence scaled up for the Stage-3 material generation. Detailed experimental procedure followed for the preparation of Stage-3 is given below.

### Experimental Procedure for Stage-3 Synthesis

To a solution of Stage-2 (1 eq.) in Dry DCM (20.0 vol.), DMAP (1.0 eq.), DCC (1.0 eq.) and lipoic acid (1.0 eq.) were added at 20-30° C. U/N₂ atmosphere and stirred at same temperature for 18 h. Reaction was monitored by TLC. Charged water (20.0 vol.), layers separated. Organic layer was washed with water (20 vol.) and concentrated to dryness. The crude compound was purified by silica gel column chromatography using hexane/EtOAc as eluents to result in the product as off white solid.

### Synthesis of Stage-4 of CLX-SYN-G162D-C02:

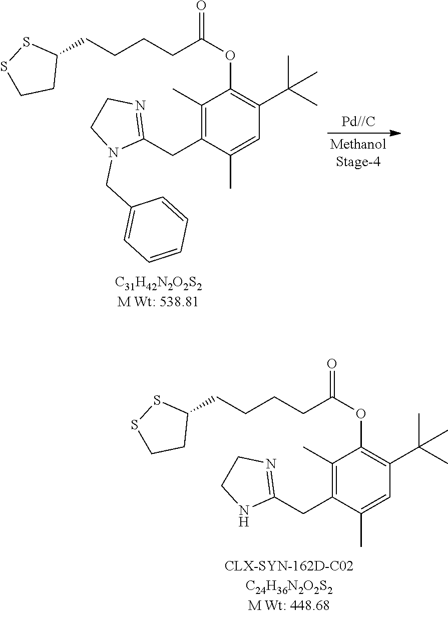 [SMILES: Cc1cc(C(C)(C)C)c(OC(=O)CCCC[C@@H]2CCSS2)c(C)c1CC1=NCCN1Cc1ccccc1 ; Cc1cc(C(C)(C)C)c(OC(=O)CCCC[C@@H]2CCSS2)c(C)c1CC1=NCCN1]

### Feasibility of Stage-4 De Benzylation Reaction

The Stage-4 de benzylation reaction was initially attempted under regular conditions using 10% Pd/C (50% wet) (50% w/w) in methanol as solvent under hydrogen atmosphere. The Stage-1 reaction i.e. formation of Oxymetazoline Hydrochloride was achieved successfully using aqueous ammonia solution and resulted product in high yield (>90%). The Stage-2 benzyl protection reaction of Stage-1 product was also achieved in about 50% yield using sodium hydride and benzyl chloride in DMF as solvent. The required Stage-3 product formation was observed in presence of DCC and DMAP in DCM as solvents resulted the required product.

### Example 3: CLX-SYN-G162D-C04

### IUPAC Name of CLX-SYN-G162D-C04: 3-(((1R,5S)-8-methyl-8-azabicyclo[3.2.1]octan-3-yl)oxy)-3-oxo-2-phenylpropyl 5-((R)-1,2-dithiolan-3-yl)pentanoate

### Chemical Structure of CLX-SYN-G162D-C04:

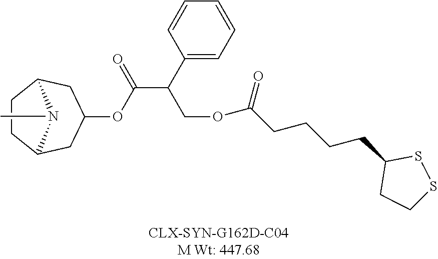

### Synthesis of CLX-SYN-G162D-C04

Synthesis of CLX-SYN-G162D-C04 is a one step process involving atropine and (R)-lipoic acid as main key raw materials.

### Scheme for Synthesis of CLX-SYN-G162D-C04

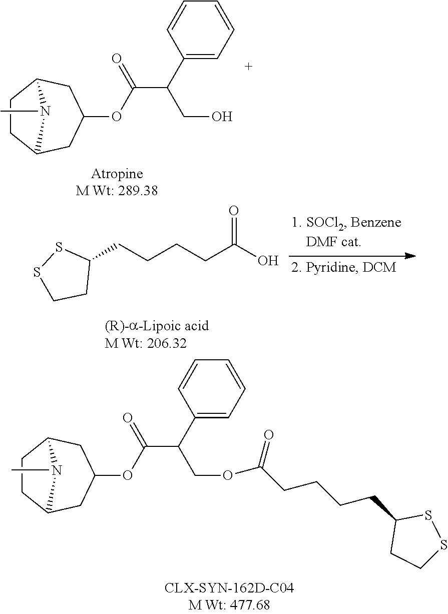

The Synthesis of CLX-SYN-G162D-C04 involves coupling of lipoic acid chloride with atropine in presence of pyridine in Dichloromethane (DCM) as solvent. Detailed experimental procedure provided in the below section.

### Synthetic Scheme of Stage-I

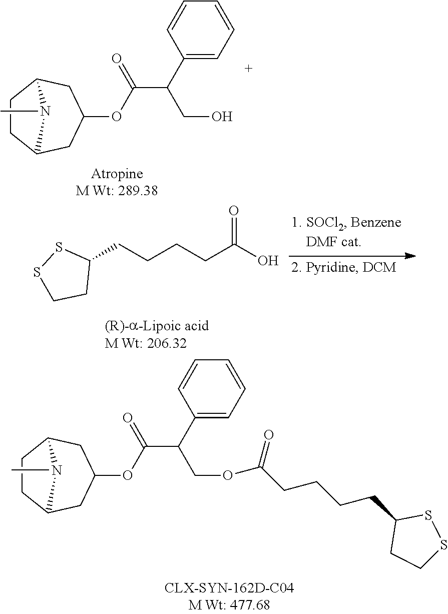

### Experimental Procedure

To a solution of (R)-lipoic acid (1.5 eq.) in benzene (3.0 vol.), DMF (0.1 vol.) followed by thionyl chloride (1.3 eq.) were added at 25-30° C. under N2 atmosphere. RM stirred for 2 h at the same temperature. Solvent was evaporated to dryness and the residue was kept aside under N2 atmosphere.

To a solution of Atropine (1.0 eq.) and pyridine (10.0 eq.) in DCM (10.0 vol.) at 0-5° C., the above prepared acid chloride solution in Benzene (4 vol.) was added under N₂ atmosphere. Reaction mixture stirred for 1-2 h at the same temperature. Reaction monitored by TLC. Reaction mass was quenched with water (8 vol.) and layers were separated. Organic layer was washed with saturated aq. NaHCO3 solution (4 vol.). Organic layer concentrated and purified by silica gel column chromatography using 5% MeOH/DCM as eluents to obtain the product as gummy liquid. The liquid compound was slurred in MTBE (3.0 vol.) for 1 h and the solid obtained was filtered, dried to give the compound as light cream color low melting solid (solidifies at low temperature).

### Structure Characterization Data of CLX-SYN-G162D-C04

Chemical structure of CLX-SYN-G162D-C04 has been established using various spectroscopic techniques such as IR, NMR, Mass and 13C NMR and results are summarized below.

Infrared Spectrophotometry: The FT-IR spectrum of CLX-SYN-G162D-C04 recorded in KBr pellet using FT-IR spectrophotometer and exhibits following IR absorption bands characteristic of its chemical structure as tabulated below.

IR (cm⁻¹): Characteristic Absorption Bands

| Position of the Functional group | Wave number (cm⁻¹) |
|---|---|
| C—H(aromatic) | 2955 |
| —C═O (ester) | 1734 |
| C═C (Aromatic) | 1667 |
| O═C—O (C—O of ester) | 1156 |
| —C—N | 1244 |
| —C—H | 734 |

¹H NMR (400 MHz, DMSO-d₆) δ ppm:

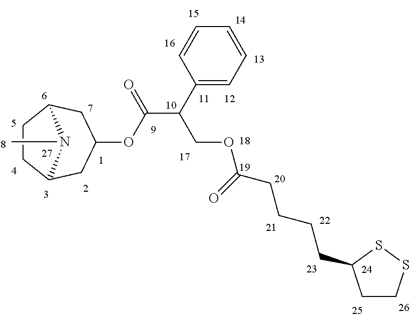

### Assignment of Chemical Shifts to Protons

| | Chemical shift (δ) with multiplicity |
|---|---|
| Position of the proton | ppm |
| 21 (2H) | 1.25-1.35 (2H, m) |
| 22 (2H), 4 (2H), 7 (2H) | 1.39-1.68 (6H, m) |
| 23 (1H) | 1.68-1.75 (1H, d) |
| 5 (2H), 23 (1H) | 1.80-2.00 (4H, m) |
| 25 (1H) | 2.00-2.10 (1H, m) |
| 20 (2H) | 2.25 (2H, t) |
| 2 (2H), 25 (1H) | 2.35-2.45 (3H, m) |
| 8 (3H) | 2.58 (3H, br s) |
| 26 (2H) | 3.00-3.30 (2H, m) |
| 24 (1H) | 3.50-3.61 (1H, m) |
| 3 (1H) | 3.68 (1H, br s) |
| 6 (1H) | 3.79 (1H, br s) |
| 10 (1H) | 4.10 (1H, dd) |
| 17 (1H) | 4.33 (1H, dd) |
| 17 (1H) | 4.56 (1H, dd) |
| 1 (1H) | 4.90-5.00 (1H,brm) |
| 12-16 (5H) | 7.30-7.40 (5H, m) |

¹³C NMR (100 MHz, DMSO-d₆):

### Assignment of Chemical Shifts to Carbon Atoms

| Position of the Carbon atoms | Chemical shift, ppm |
|---|---|
| C-4, C-5 | 24.08 |
| C-21, C-22 | 27.91 |
| C-20, C-23 | 33.14 |
| C-2, C-7 | 33.94 |
| C-25, C-8 | 38.07 |
| C-26 | 48.55 |
| C-10 | 49.77 |
| C-24 | 55.97 |
| C-17 | 60.87 |
| C-3, C-6 | 63.75 |
| C-1 | 65.15 |
| C-14 | 127.82 |
| C-13,C-15 | 128.16 |
| C-12, C-16 | 128.79 |
| C-11 | 135.14 |
| C-19 | 169.90 |
| C-9 | 172.41 |

MS: 478 (M−H)+ve mode.

CLX-SYN-G162D-C04 was synthesized in one step starting from Atropine and (R)-Lipoic acid in about 10% yield with >97% purity by HPLC.

### Example 4: CLX-SYN-G162D-C06

### IUPAC name of CLX-SYN-G162D-C06: (1S,5S)-3-((3-hydroxy-2-phenylpropanoyl)oxy)-8-methyl-8-azabicyclo[3.2.1]octan-8-ium (R)-5-(1,2-dithiolan-3-yl)pentanoate

### Chemical Structure of CLX-SYN-G162D-C06:

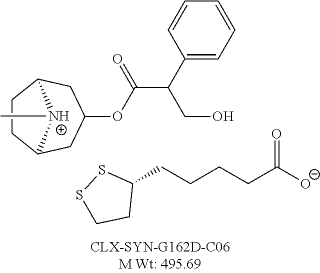 [SMILES: C[N@H+]1C2CC[C@H]1CC(OC(=O)C(CO)c1ccccc1)C2 ; O=C([O-])CCCC[C@@H]1CCSS1]

### Synthesis of CLX-SYN-G162D-C06

Synthesis of CLX-SYN-G162D-C06 is a one step process involving Atropine and (R)-Lipoic acid as main key raw materials.

### Scheme for Synthesis of CLX-SYN-G162D-C06

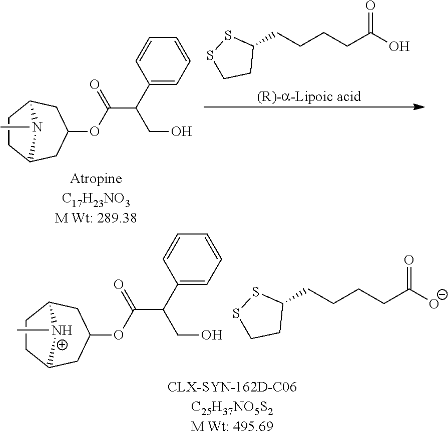

The synthesis of CLX-SYN-G162D-C06 involves treatment of atropine with (R)-lipoic acid in ethanol. Detailed experimental procedure is provided in the below sections.

### Synthetic Scheme of Stage-I

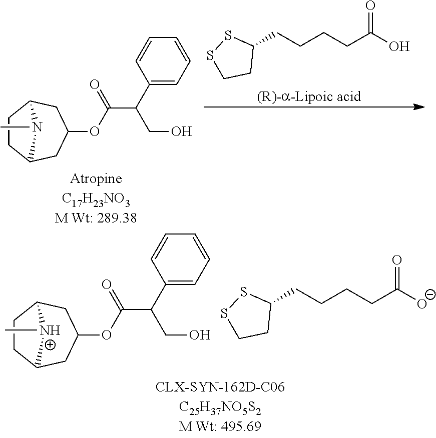

### Experimental Procedure

To a stirred solution of Atropine (1.0 eq.) in thanol (10 vol.), (R)-lipoic acid (1.0 eq.) was added and the reaction mixture was stirred for 17.0 h at 25-30° C. The reaction mixture was concentrated under reduced pressure to remove solvent, and resulted in the required compound as pale yellow color liquid.

### Structure Characterization Data of CLX-SYN-G162D-C06

Chemical structure of CLX-SYN-G162D-C06 has been established using various spectroscopic techniques such as IR, NMR, Mass and 13C NMR and results are summarized below.

Infrared Spectrophotometry: The FT-IR spectrum of CLX-SYN-G162D-C06 recorded using Sodium chloride cells in FT-IR spectrophotometer and exhibits following IR absorption bands characteristic of its chemical structure as tabulated below.

IR (cm⁻¹): Characteristic Absorption Bands:

| Position of the | |
|---|---|
| Functional group | Wave number (cm⁻¹) |
| C—H (aromatic) | 2933 |
| O—H(Alcohol) | 3324 |
| —C═O | 1730 |
| C═C (Aromatic) | 1573 |
| O═C—O (C—O of ester) | 1162 |
| —C—N | 1222 |
| —C—H | 754 |

¹H NMR (400 MHz, CDCl₃) δ ppm:

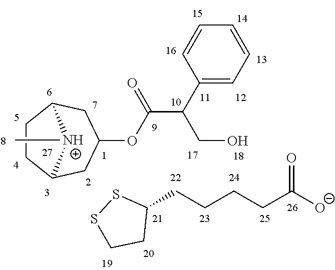 [SMILES: C[N@H+]1C2CC[C@H]1CC(OC(=O)C(CO)c1ccccc1)C2 ; O=C([O-])CCCC[C@@H]1CCSS1]

### Assignment of Chemical Shifts to Protons:

| | Chemical shift (δ) with |
|---|---|
| Position of the proton | multiplicity ppm |
| 23 (2H) | 1.20-1.26 (2H, m) |
| 22 (2H) | 1.29-1.35 (2H, m) |
| 24 (2H) | 1.40-1.58 (2H, m) |
| 5 (2H), 4 (2H), 25 (2H) | 1.60-1.85 (6H, m) |
| 2 (2H), 7 (2H) | 1.85-2.10 (4H, m) |
| 10 (1H) | 2.10-2.35 (1H, m) |
| 8 (3H), 20 (2H) | 2.40-2.70 (5H, m) |
| 3 (1H), 6 (1H) | 3.10-3.20 (2H, m) |
| 27 (1H) | 3.35-3.45 (1H, br s) |
| 19 (2H) | 3.50-3.61 (2H, m) |
| 17 (2H), 21 (1H) | 3.70-3.90 (3H, m) |
| 1 (1H) | 4.10-4.30 (1H, m) |
| 18 (1H) | 5.12 (1H, br s) |
| 12-16 (5H) | 7.20-7.40 (5H, m) |

¹³C NMR (100 MHz, DMSO-d₆):

### Assignment of Chemical Shifts to Carbon Atoms:

| Position of the Carbon atoms | Chemical shift, ppm |
|---|---|
| C-4 | 24.10 |
| C-5 | 24.63 |
| C-25 | 25.54 |
| C-22 | 29.09 |
| C-2 | 34.19 |
| C-7 | 34.37 |
| C-24 | 34.68 |
| C-23 | 36.24 |
| C-21 | 38.39 |
| C-20 | 40.17 |
| C-10 | 54.28 |
| C-19 | 56.52 |
| C-8 | 58.26 |
| C-3 | 60.11 |
| C-6 | 60.23 |
| C-17 | 63.92 |
| C-1 | 66.14 |
| C-14 | 127.87 |
| C-13,C-15 | 128.03 |
| C-12, C-16 | 128.96 |
| C-11 | 135.39 |
| C-26 | 171.87 |
| C-9 | 179.34 |

MS: 290 (M+H)+ve mode & 205 (M−H)-ve mode.

To conclude, CLX-SYN-G162D-C06 was synthesized in one step from atropine and (R)-lipoic acid. The isolated salt compound was found to be liquid and various attempts for the solid isolation of the final compound, i.e. atropine lipoate, were unsuccessful, which could be attributed to the formation of diastereomeric salts of Atropine Lipoate during the reaction.

### EQUIVALENTS

The present disclosure provides among other things compositions and methods for treating anorectal diseases and their complications. While specific embodiments of the subject disclosure have been discussed, the above specification is illustrative and not restrictive. Many variations of the systems and methods herein will become apparent to those skilled in the art upon review of this specification. The full scope of the claimed systems and methods should be determined by reference to the claims, along with their full scope of equivalents, and the specification, along with such variations.

### INCORPORATION BY REFERENCE

All publications and patents mentioned herein, including those items listed above, are hereby incorporated by reference in their entirety as if each individual publication or patent was specifically and individually indicated to be incorporated by reference. In case of conflict, the present application, including any definitions herein, will control.

What is claimed is:

1.-5. (canceled)

6. A compound of formula VI 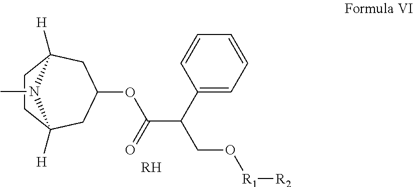
and pharmaceutically acceptable salts, hydrates, solvates, prodrugs, enantiomers, stereoisomers and derivatives thereof,
wherein, RH independently represents
NULL, Cl—, Na+, pyridoxine, R-lipoic acid, methyl cellulose, glutamic acid, aspartic acid, valproic acid, prostaglandin F2a, dopamine, morphine, loperamide, b-blockers, sodium valproate, codeine phosphate, N-acetyl cysteine, cysteine, 5-Hydroxytryptamine, Zn+2, Mg+2, Mn+2, I—, Ag+2, Na, K, mesalamine, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, benzenesulfonic acid, benzoic acid, camphoric acid, camphor-10-sulfonic acid, capric acid (decanoic acid), caproic acid (hexanoic acid), carbonic acid, cinnamic acid, citric acid, cyclamic acid, dodecylsulfuric acid, ethane-1,2-disulfonic acid, ethanesulfonic acid, formic acid, galactaric acid, gentisic acid, glucoheptonic acid, gluconic acid, glucuronic acid, glutaric acid, glycerophosphoric acid, glycolic acid, hippuric acid, hydrobromic acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid, salicylic acid, sebacic acid, stearic acid, succinic acid, sulfuric acid, tartaric acid, thiocyanic acid, toluenesulfonic acid, undecylenic acid, omega 3 fatty acids, omega 6 fatty acids, furoate, methyl furoate, ethyl furoate, aminocaproic acid, caprilic acid, alpha lipoic acid, myristic acid, myristoleic acid, palmitoleic acid, elaidic acid, linoleic acid, linolenic acid, linolelaidic acid, arachidonic acid, 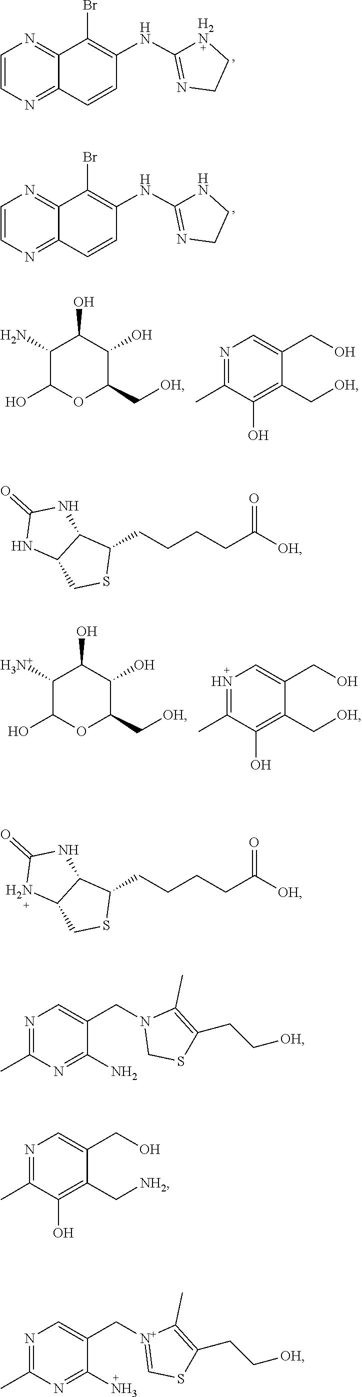 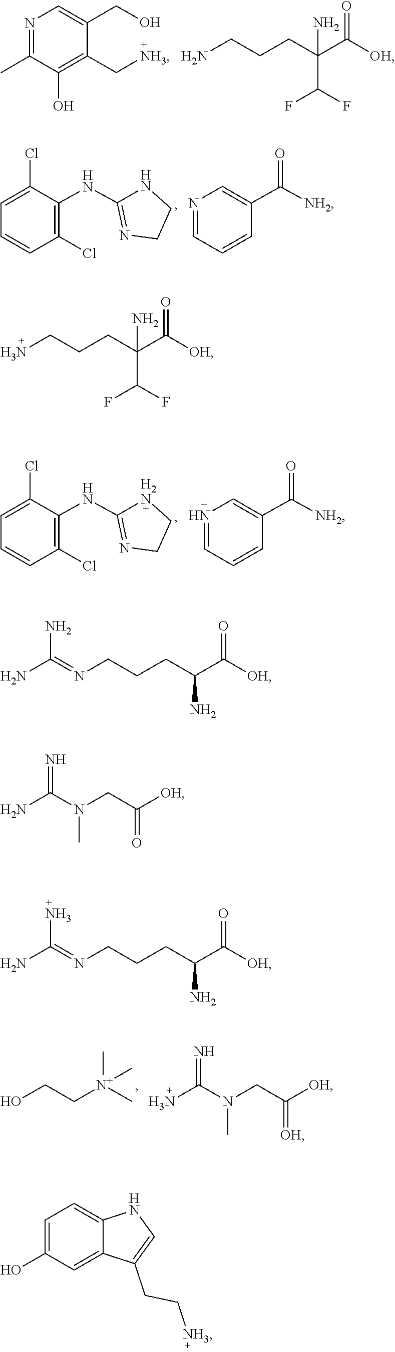 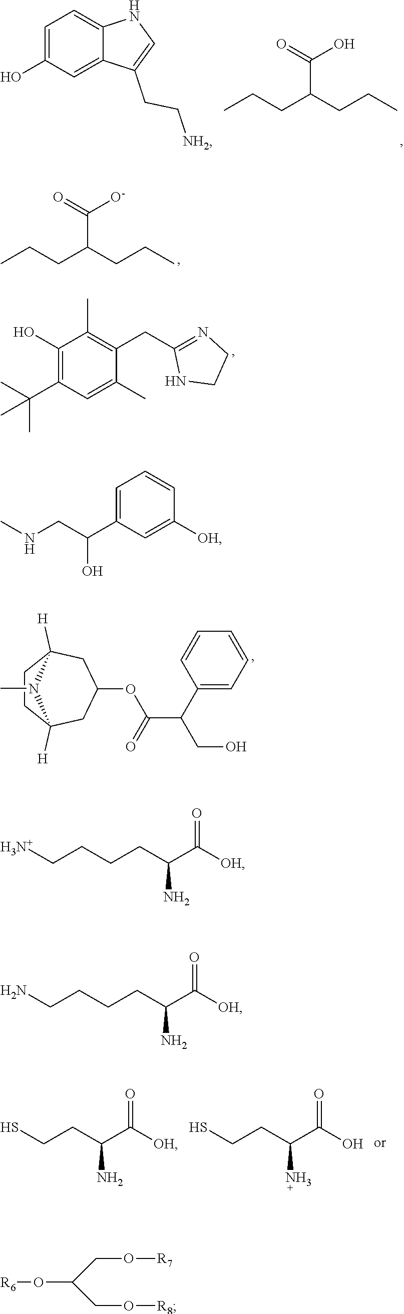
and wherein, within the proviso
each R₆, R₇ and R₈ independently represents 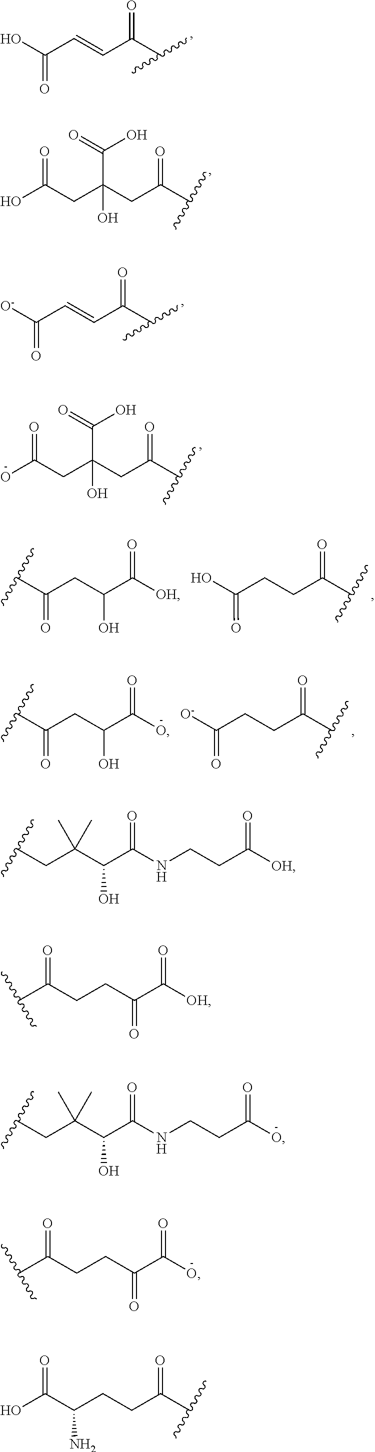 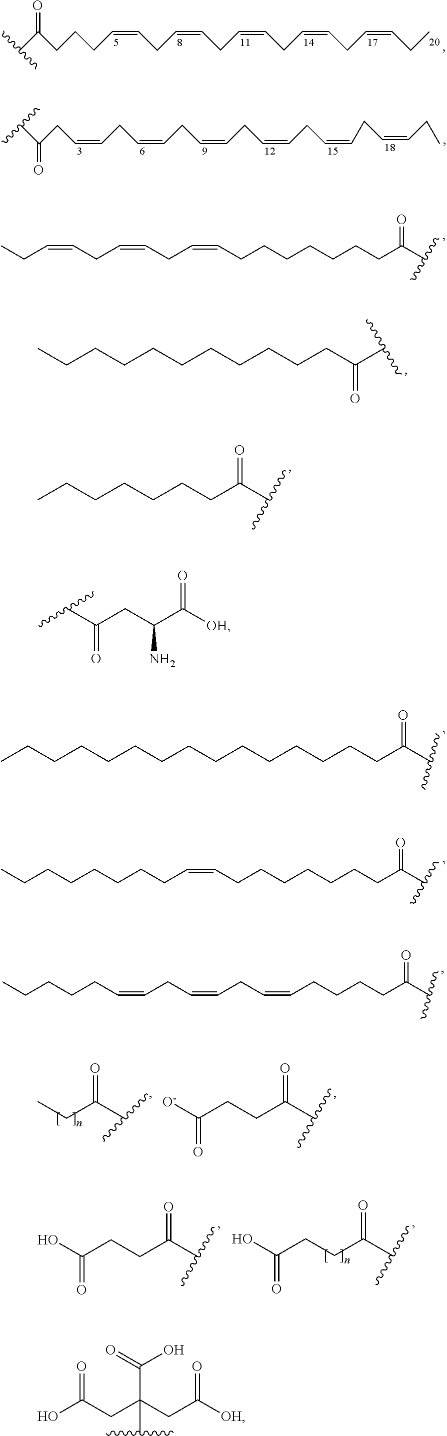 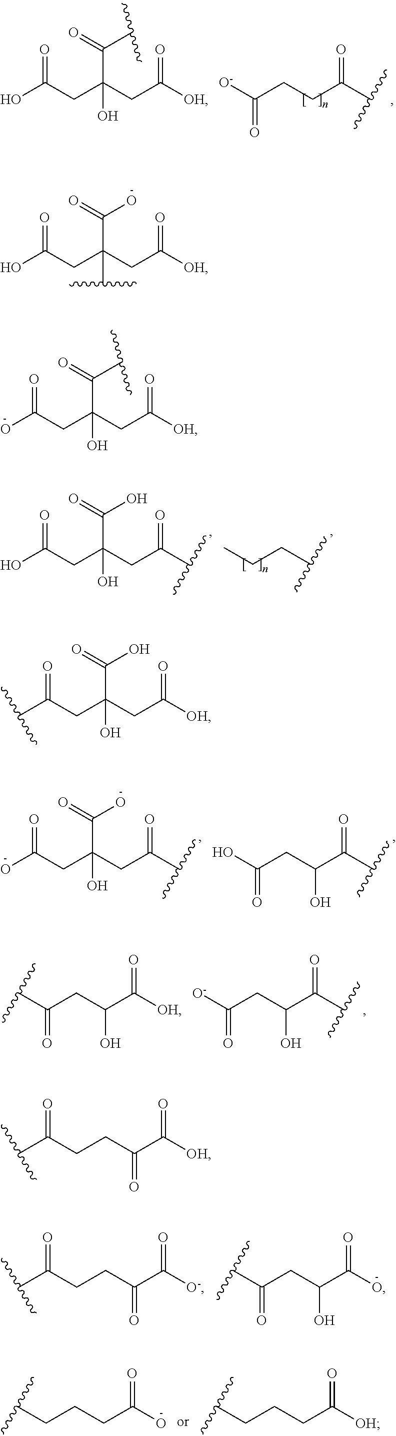
and wherein, within the proviso, n=0 to 80;
R₁ represents null, 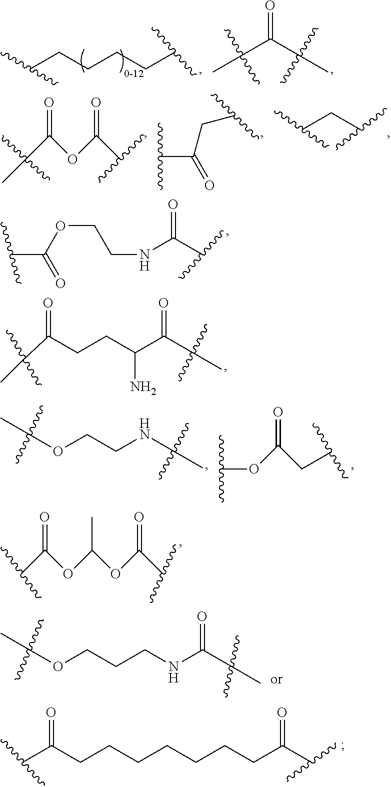
R₂ represents

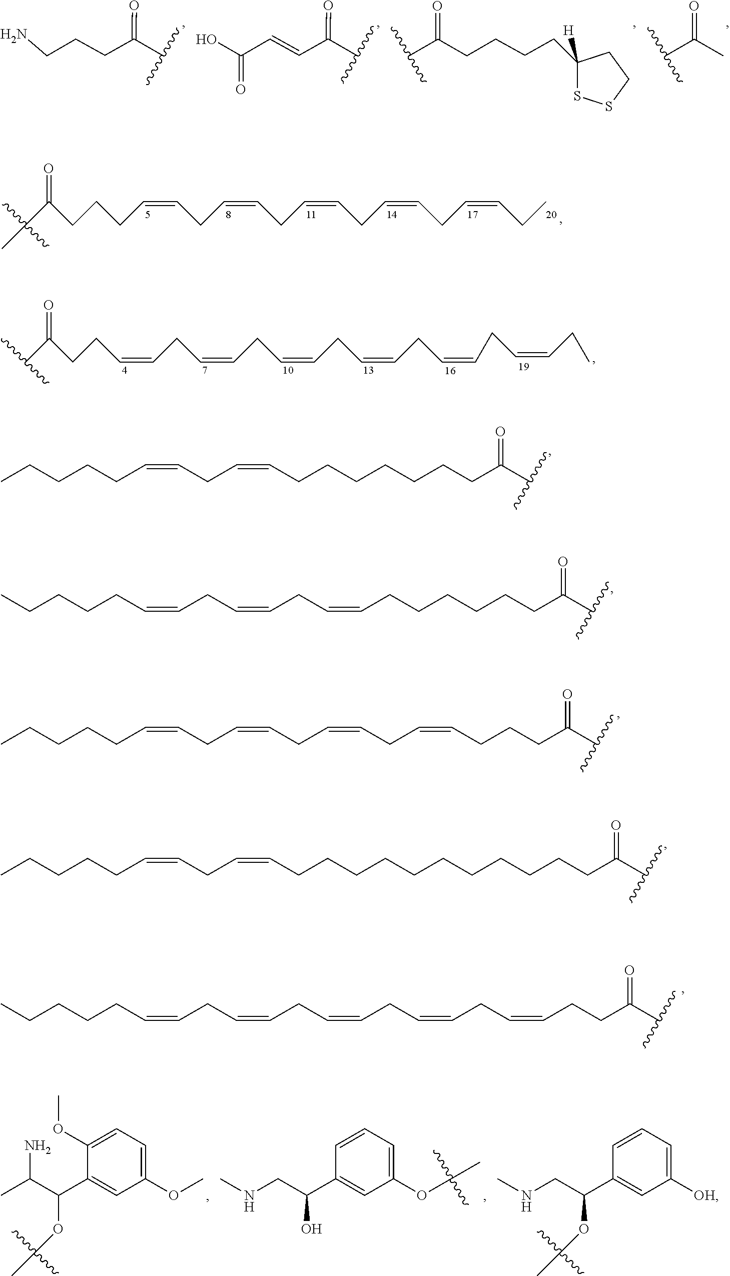

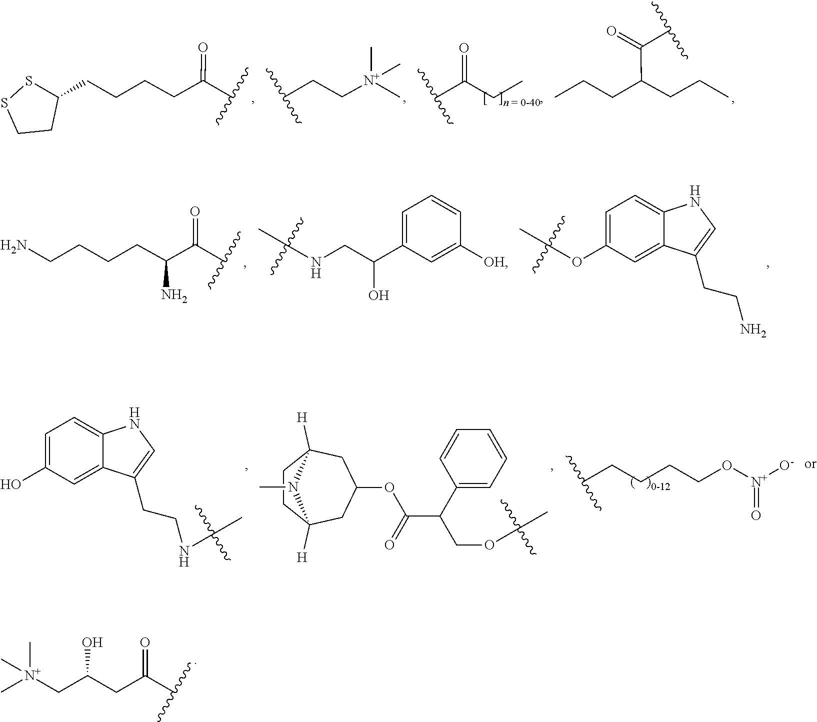

7. A compound of formula VII 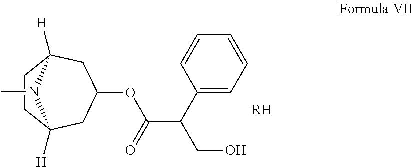
and pharmaceutically acceptable salts, hydrates, solvates, prodrugs, enantiomers, stereoisomers and derivatives thereof,
wherein,
RH independently represents
NULL, pyridoxine, R-lipoic acid, methyl cellulose, aspartic acid, valproic acid, prostaglandin F2a, dopamine, morphine, loperamide, b-blockers, sodium valproate, codeine phosphate, N-acetyl cysteine, cysteine, 5-Hydroxytryptamine, Zn+2, Mg+2, Mn+2, I—, Ag+2, Na, K, mesalamine, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid, camphoric acid, camphor-10-sulfonic acid, capric acid, caproic acid, cinnamic acid, cyclamic acid, dodecylsulfuric acid, formic acid, galactaric acid, gentisic acid, glucoheptonic acid, gluconic acid, glucuronic acid, glutaric acid, glycerophosphoric acid, glycolic acid, hippuric acid, hydrobromic acid, isobutyric acid, lauric acid, malonic acid, methanesulfonic acid, naphthalene-1,5-disulfonic acid, naphthalene-2-sulfonic acid, nicotinic acid, oleic acid, oxalic acid, palmitic acid, proprionic acid, pyroglutamic acid, sebacic acid, thiocyanic acid, toluenesulfonic acid, undecylenic acid, omega 3 fatty acids, omega 6 fatty acids, furoate, methyl furoate, ethyl furoate, aminocaproic acid, caprilic acid, alpha lipoic acid, myristic acid, myristoleic acid, palmitoleic acid, elaidic acid, linoleic acid, linolenic acid, linolelaidic acid, arachidonic acid, 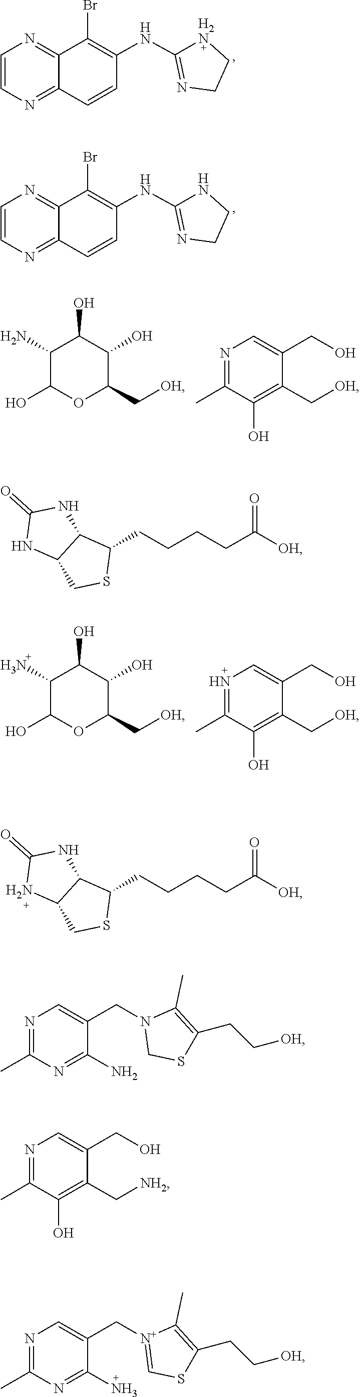 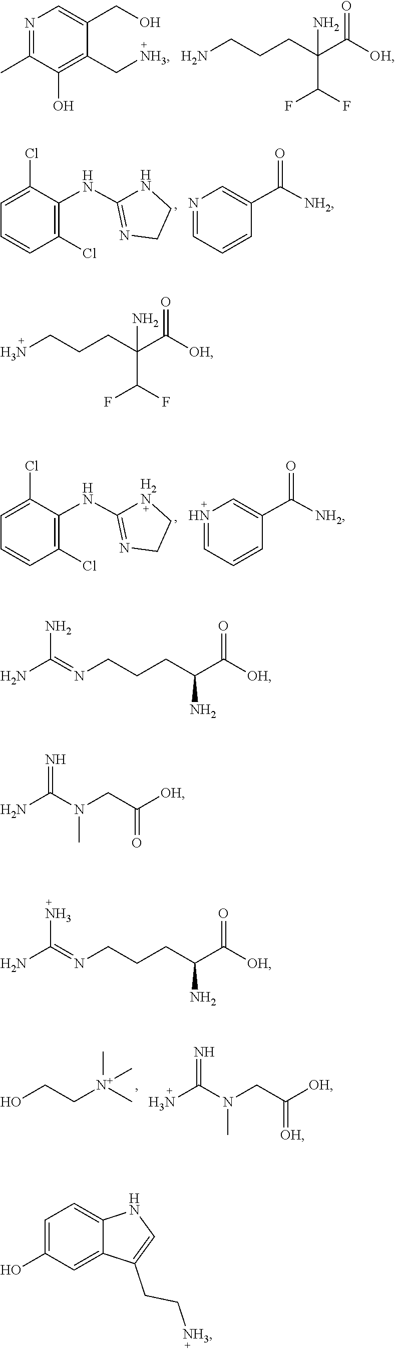 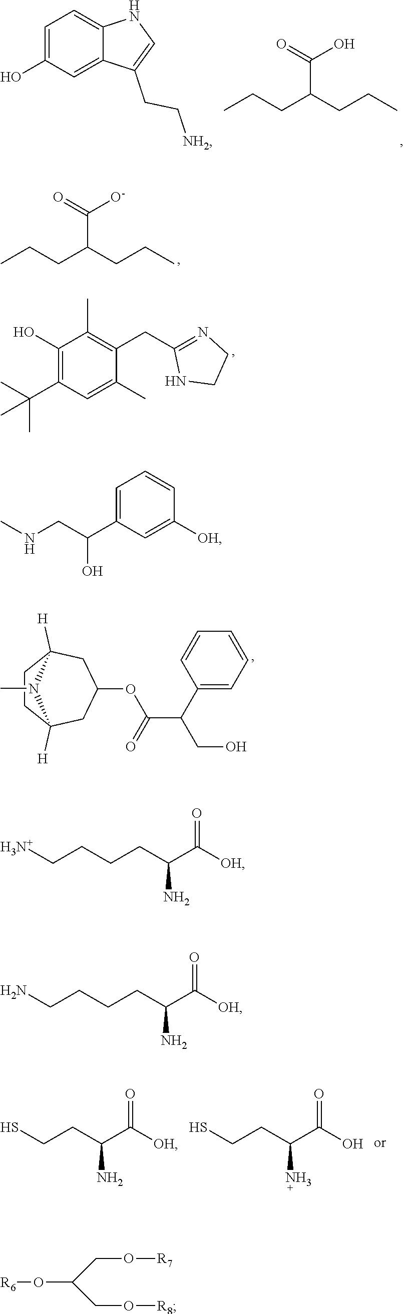
and wherein, within the proviso
each R₆, R₇ and R₈ independently represents 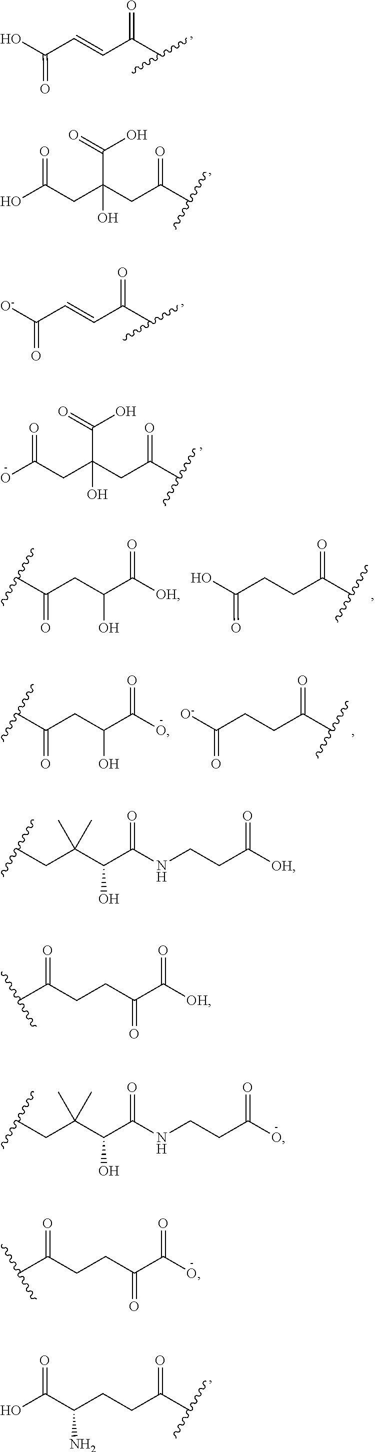 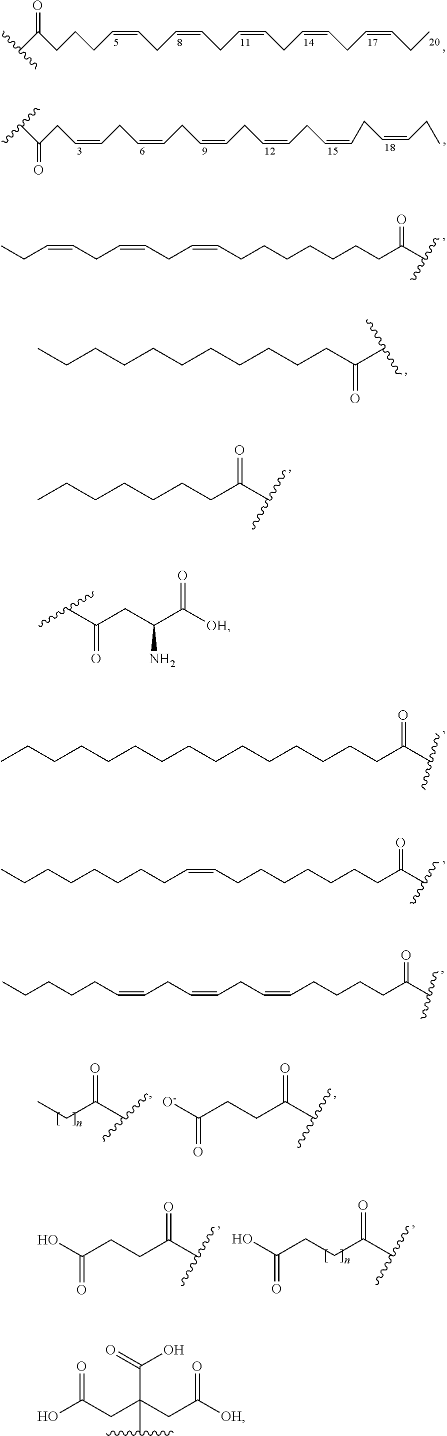 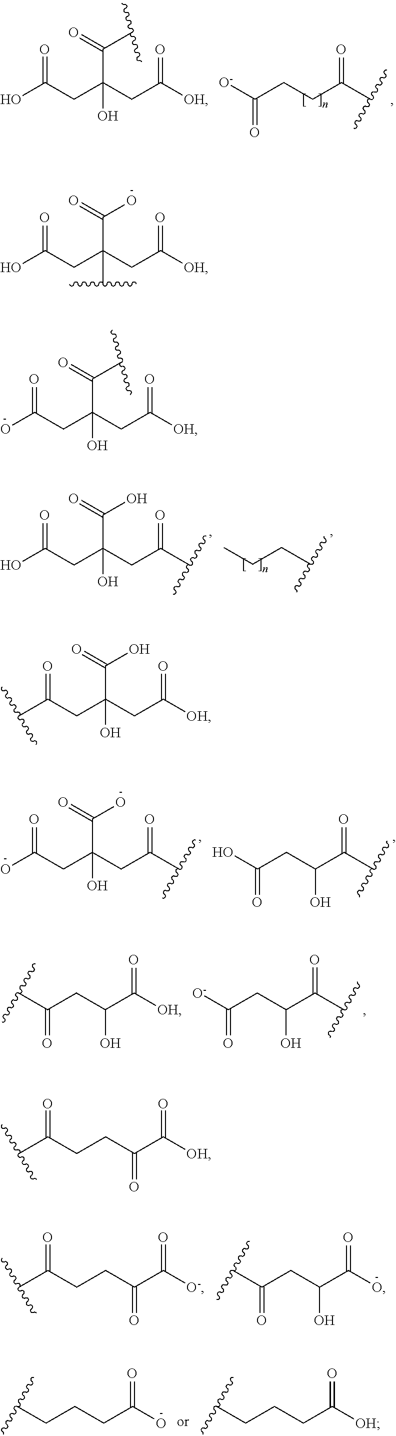
and wherein, within the proviso, n=0 to 80.

8.-12. (canceled)

13. A pharmaceutical composition comprising a compound of claim 6, and a pharmaceutically acceptable carrier.

14. A pharmaceutical composition comprising a compound of claim 7, and a pharmaceutically acceptable carrier.

15.-19. (canceled)

20. The pharmaceutically composition of claim 13, said compound is formulated to treat a patient in need with an effective amount of said pharmaceutical compound by administering the patient in need by oral administration, rectal administration, buccal administration, subdermal administration, parenteral administration, systemic administration, delayed release, sustained release administration, or transdermal administration.

21. The pharmaceutical composition of claim 14, wherein said compound is formulated to treat a patient in need with an effective amount of said pharmaceutical compound by administering the patient in need by oral administration, rectal administration, buccal administration, subdermal administration, parenteral administration, systemic administration, delayed release, sustained release administration, or transdermal administration.

22.-26. (canceled)

27. The pharmaceutical composition of claim 20, wherein said composition is formulated for the treatment of anal and rectal disorders

28. The pharmaceutical composition of claim 21, wherein said composition is formulated for the treatment of anal and rectal disorders.

29. (canceled)

30. A compound of claim 6, wherein said compound is formulated for the treatment of anal and rectal disorders.

31. A compound of claim 7, wherein said compound is formulated for the treatment of anal and rectal disorders.

32. A compound selected from the group of structures consisting of

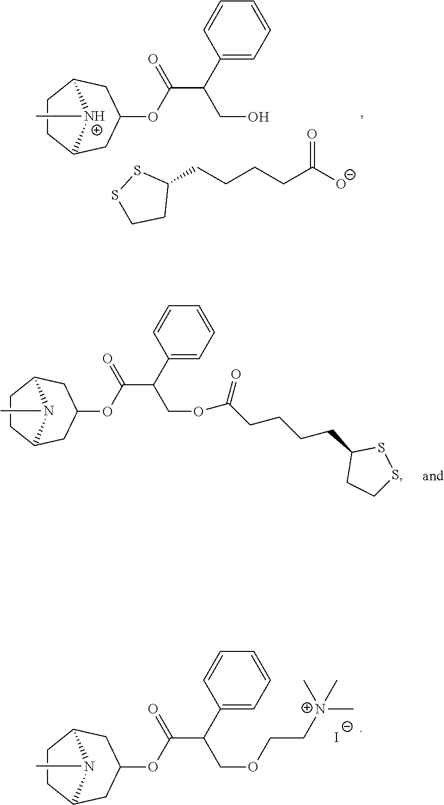

33. A pharmaceutical composition comprising a compound of claim 32, and a pharmaceutically acceptable carrier.

34. The pharmaceutical composition of claim 33, wherein said compound is formulated to treat a patient in need with an effective amount of said pharmaceutical compound by administering the patient in need by oral administration, rectal administration, buccal administration, subdermal administration, parenteral administration, systemic administration, delayed release, sustained release administration, or transdermal administration.

35. A pharmaceutical composition of claim 34, wherein said composition is formulated for the treatment of anal and rectal disorders.

36. A compound of claim 32, wherein said compound is formulated for the treatment of anal and rectal disorders.
